(19) United States
(12) Patent Application Publication
(10) Pub. No.: US 2018/0327724 A1
(43) Pub. Date: Nov. 15, 2018

(54) METHOD FOR PRODUCING HEAVY CHAIN AMINOCARBOXYLIC ACID

(71) Applicant: KOREA RESEARCH INSTITUTE OF BIOSCIENCE AND BIOTECHNOLOGY, Daejeon (KR)

(72) Inventors: Jung Oh AHN, Daejeon (KR); Hong Weon LEE, Daejeon (KR); Gyu Yeon PARK, Daejeon (KR); Min Jeong JANG, Daejeon (KR); Woo Young JEON, Daejeon (KR)

(73) Assignee: KOREA RESEARCH INSTITUTE OF BIOSCIENCE AND BIOTECHNOLOGY, Daejeon (KR)

(21) Appl. No.: 15/771,812

(22) PCT Filed: Oct. 27, 2016
(86) PCT No.: PCT/KR2016/012172

§ 371 Date: Jun. 22, 2018

(30) Foreign Application Priority Data
Oct. 27, 2015 (KR) 10-2015-0149254
Oct. 27, 2016 (KR) 10-2016-0141019

(51) Int. Cl. C12N 9/10; C12N 15/81; C12P 13/00

(52) U.S. Cl. CPC C12N 9/1096; C12P 13/005; C12N 15/815; C12Y 206/01

(57) ABSTRACT

The present invention relates to a method for producing a medium chain aminocarboxylic acid and, more particularly, to a recombinant microorganism in which a fatty aldehyde dehydrogenase gene in ω-oxidative metabolic pathway and a β-oxidative metabolic pathway-related gene are deleted and a ω-transaminase gene is introduced, and a method for producing a medium chain aminocarboxylic acid by culturing the recombinant microorganism. The recombinant microorganism of the present invention can prevent additional oxidation of fatty aldehyde and β-oxidative metabolism and also produce a medium chain aminocarboxylic acid, such as 12-aminodecane, as a raw material of nylon 12 from a substrate such as fatty acid with a high yield by introducing an amine group into a terminal thereof.

### TECHNICAL FIELD

The present invention relates to a method for producing a medium chain aminocarboxylic acid, and more particularly, to a method for producing a medium chain aminocarboxylic acid from a fatty acid by culturing a recombinant microorganism from which a fatty aldehyde dehydrogenase (or fatty alcohol dehydrogenase) gene in an ω-oxidative metabolism pathway and β-oxidative metabolism pathway-related genes are deleted and into which an ω-transaminase gene is also introduced.

### BACKGROUND ART

Bioplatform compounds are produced through biological or chemical conversion on the basis of biomass-derived raw materials, and thus have been used for synthesis of polymeric monomers, new materials, and the like.

Among the bioplatform compounds, a medium chain aminocarboxylic acid is a material used as a monomer for polyamides. The polyamides are classified into aliphatic polyamides, aromatic polyamides, and aliphatic cyclic polyamides. Representative examples of the aliphatic polyamides includes Nylon 12, Nylon 6, and Nylon 66, and the aromatic polyamides have an aromatic framework introduced therein in order to further improve heat resistance, and are also known under the name of aramid.

Since the 1940's, nylon is a representative engineering plastic material whose demand and use have increased steadily due to high crystallinity, mechanical strength and thermal stability, excellent wear/friction resistance characteristics, and the like. Also, there has been continuous research conducted in various fields to improve the thermal and mechanical properties of nylon. Among theses, there is research conducted to impregnate wax and graphite so as to improve wear resistance of nylon. In addition, there is ongoing research conducted to improve the physical properties of nylon through the crosslinking of polymers. Among the types of nylon, Nylon 12 synthesized through the polycondensation of 12-aminododecanoic acid exhibits low specific gravity, excellent low-temperature characteristics and wear resistance and high weather resistance due to the insignificant effects of ultraviolet rays, and also has a very short —CH₂— chain length, compared to other nylon resins (i.e., Nylons 6, 66). Therefore, because Nylon 12 has a low probability of having a hydrogen bond of H₂O to an amide functional group when present in the same weight, Nylon 12 may serve to prevent the degradation of mechanical strength caused by moisture absorption, which is one of the biggest drawbacks of the nylon resins, which makes it possible to widely apply it to materials for automobile parts, aircraft materials, heat-resistant special fibers, and the like (Beomsik, Shin, et al., Polymer, 35(1):30-34, 2011).

Production of medium chain aminocarboxylic acids such as 12-aminododecanoic acid may be carried out using biological methods through chemical synthesis or microbial fermentation. In this case, the use of such biological methods requires the development of novel strains and the optimization of fermentation processes using metabolic engineering technology.

In the prior art, a microorganism which harbors both a β-oxidative metabolism pathway and an ω-oxidative metabolism pathway may be used as the strain capable of producing a medium chain aminocarboxylic acid. For example, a method for producing ω-aminododecanoic acid in Escherichia coli is known (US 2010/0324257 A1). However, because the medium chain aminocarboxylic acid is prepared by further introducing a process of transferring an amine group to a medium chain aldehyde carboxylic acid, the medium chain aminocarboxylic acid has a drawback in that it may not be produced with high yield when it is produced using the microorganism.

### DISCLOSURE

### Technical Problem

Therefore, it is an object of the present invention to provide a recombinant microorganism from which a fatty aldehyde dehydrogenase gene in an ω-oxidative metabolism pathway and β-oxidative metabolism pathway-related genes are deleted and into which an ω-transaminase gene is also introduced, and a method for producing a medium chain aminocarboxylic acid from a fatty acid by culturing the recombinant microorganism.

### Technical Solution

To solve the above problems, according to an aspect of the present invention, there is provided a recombinant microorganism from which a fatty aldehyde dehydrogenase gene in an ω-oxidative metabolism pathway and β-oxidative metabolism pathway-related genes are deleted and into which an ω-transaminase gene is also introduced.

According to an embodiment of the present invention, the fatty aldehyde dehydrogenase gene and the β-oxidative metabolism pathway-related genes are preferably deleted from all homologous genes present in the microorganism, but the present invention is not limited thereto. According to another embodiment of the present invention, the fatty aldehyde dehydrogenase gene and the β-oxidative metabolism pathway-related genes are preferably deleted from some of the homologous genes present in the corresponding microorganism, but the present invention is not limited thereto.

According to an embodiment of the present invention, the fatty aldehyde dehydrogenase gene may be a gene selected from the group consisting of FALDH1, FALDH2, FALDH3, and FALDH4 genes, but the present invention is not limited thereto.

According to an embodiment of the present invention, the β-oxidative metabolism pathway-related genes may be an acyl-CoA oxidase gene, but the present invention is not limited thereto. According to preferred embodiments of the present invention, the acyl-CoA oxidase gene may be selected from the group consisting of ACO1, ACO2, ACO3, ACO4, ACO5, and ACO6 genes, but the present invention is not limited thereto.

According to an embodiment of the present invention, the microorganism may be a yeast or Escherichia coli, but the present invention is not limited thereto. According to preferred embodiments of the present invention, the yeast may be selected from the group of the yeast consisting of Yarrowia sp., Saccharomyces sp., Pichia sp., and Candida sp., but the present invention is not limited thereto. According to other preferred embodiments of the present invention, the Yarrowia sp. yeast may be Yarrowia lipolytica, but the present invention is not limited thereto.

According to another aspect of the present invention, there is provided a method for producing a medium chain aminocarboxylic acid, which comprises (1) preparing the recombinant microorganism from which a fatty aldehyde dehydrogenase gene in an ω-oxidative metabolism pathway and β-oxidative metabolism pathway-related genes are deleted and into which an ω-transaminase gene is also introduced; and (2) treating the recombinant microorganism with a substrate to culture the recombinant microorganism.

According to an embodiment of the present invention, the substrate may include a fatty acid, but the present invention is not limited thereto. According to preferred embodiments of the present invention, the fatty acid and medium chain aminocarboxylic acid may have 5 to 30 carbon atoms, preferably 6 to 20 carbon atoms, and more preferably 8 to 16 carbon atoms, but the present invention is not limited thereto. According to other preferred embodiments of the present invention, the fatty acid may be dodecanoic acid, but the present invention is not limited thereto. According to other preferred embodiments of the present invention, the medium chain aminocarboxylic acid may be 12-aminododecanoic acid, but the present invention is not limited thereto.

### Advantageous Effects

A recombinant microorganism of the present invention can produce a medium chain aminocarboxylic acid, for example, 12-aminodecane used as a raw material of Nylon 12, from a substrate such as a fatty acid by deleting a fatty aldehyde dehydrogenase gene in an ω-oxidative metabolism pathway and β-oxidative metabolism pathway-related genes and introducing an ω-transaminase gene.

### DESCRIPTION OF DRAWINGS

FIG. 1 is a diagram showing types of products and related enzymes associated with ω-oxidative and β-oxidative metabolism reactions.

FIG. 2 is a diagram schematically showing a process of preparing a recombinant microorganism of the present invention from which a fatty aldehyde dehydrogenase gene associated with ω-oxidation and β-oxidative metabolism pathway-related genes are deleted and into which an ω-transaminase gene is also introduced.

FIG. 3 is a diagram schematically showing a vector containing an ura3 gene to be used as a selective marker for gene knockout to modify a strain, and a pop-out region for deleting the ura3 gene after insertion of a knock-out cassette.

FIG. 4 is a schematic diagram showing a process of constructing a knock-out cassette used to prepare a transformant microorganism according to the present invention.

FIG. 5 is a diagram schematically showing a transformation vector containing an ω-transaminase gene for the purpose of modifying a strain.

FIG. 6 is a graph illustrating types of transduced knock-out genes in the transformant microorganism according to the present invention.

FIG. 7 is a graph illustrating an amount of a medium chain aminocarboxylic acid produced from the dodecanoic acid substrate, using the transformant microorganism according to the present invention.

FIG. 8 is a graph illustrating an amount of the medium chain aminocarboxylic acid produced from the dodecanoic acid substrate, when an Y2-36 strain of the present invention is cultured in a flask.

FIG. 9 shows the GC/MS data showing that the medium chain aminocarboxylic acid is produced from the dodecanoic acid substrate in the Y2-36 strain according to the present invention.

### BEST MODE

To achieve the objectives of the present invention, the present invention provides a recombinant microorganism from which a fatty aldehyde dehydrogenase gene in an ω-oxidative metabolism pathway and β-oxidative metabolism pathway-related genes are deleted and into which an ω-transaminase gene is also introduced.

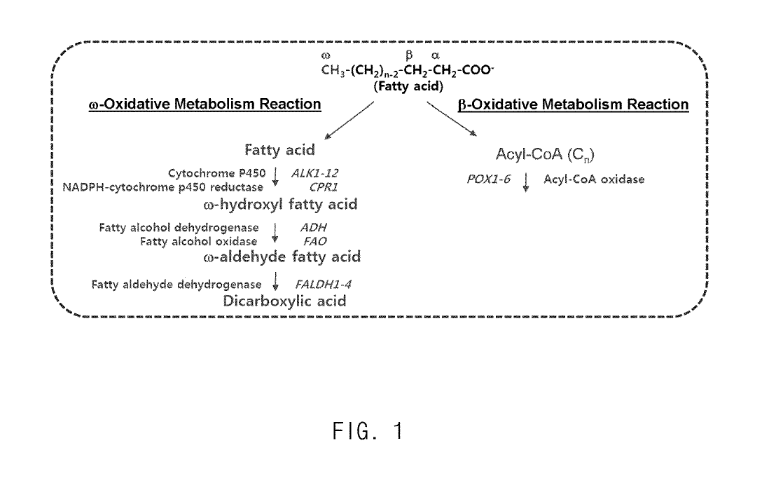

In the present invention, the term “ω-oxidation” refers to a metabolic process in which the terminal methyl group of a fatty acid is oxidized to form dicarboxylic acid, and the term “(3-oxidation” refers to a metabolic process in which a carbon atom at the β-position in a carboxyl group is oxidized to release acetyl-CoA, whereby fatty acids are gradually decomposed into fatty acids whose number of carbon atoms is reduced by two. The concept of the ω- and β-oxidations and the enzymes involved in such metabolic processes are widely known to persons having ordinary skill in the field of biochemistry. For example, when a fatty acid is used as the substrate for ω-oxidation, an ω-hydroxy fatty acid is first produced by means of an action of cytochrome P450 and an NADPH-cytochrome P450 reductase. Then, the ω-hydroxy fatty acid is converted into ω-aldehyde fatty acid by an action of a fatty alcohol dehydrogenase and a fatty alcohol oxidase, and the ω-aldehyde fatty acid is converted into dicarboxylic acid by an action of a fatty aldehyde dehydrogenase. Also, for the β-oxidation, a fatty acid whose number of carbon atoms is reduced by two is produced by an acyl-CoA oxidase (see FIG. 1).

Transaminase (TA, EC 2.6.1.X) is an enzyme which exists widely in nature and is involved in the transfer of an amine group in the nitrogen metabolism of an organism. Generally, transaminases serve to remove an amino group from one amino acid to transfer the amino group to another α-keto acid. The transaminases are used to produce optically pure non-natural amino acids and amine compounds because the transaminases have various outstanding advantages in that they exhibit wide specificity to substrates, high optical selectivity, a rapid reaction rate, and superior stability, and have no need for reproduction of coenzymes, and the like. The transaminases may be classified into five groups depending on the structures and multisequence alignments of proteins found in the Pfam database. Among these, the transaminases belonging to Group III including an ω-amino acid:pyruvate transaminase, an ornithine transaminase, a 4-aminobutyrate transaminase, and the like are referred to as ω-transaminases. Unlike the typical transaminases, the ω-transaminases perform a reaction of transferring an amine group of an amino acid- or carboxyl group-free amine compound, which contains an amine group at a position other than the α-position, to an amine receptor such as 2-ketoglutarate or pyruvate. Therefore, the ω-transaminases may be used as enzymes very useful for production of optically active amine compounds. For example, the ω-transaminases were first employed at 1990 by Celgene Co. (USA) to synthesize chiral amines. In recent years, the ω-transaminases have been importantly employed for studies on asymmetric synthesis of chiral amines and studies on improvement of kinetic resolution. In 2012, Evonik Industries AG (Germany) reported one case in which 12-oxolauric acid methyl ester is converted into 12-aminolauric acid methyl ester using an ω-transaminase of a Chromobacterium violaceum DSM30191 strain.

According to an embodiment of the present invention, the fatty aldehyde dehydrogenase gene is preferably deleted from all homologous genes present in the corresponding microorganism, but a recombinant microorganism from which some of these genes are deleted may also be applied to the present invention, when necessary.

According to an embodiment of the present invention, the fatty aldehyde dehydrogenase gene may be selected from the group consisting of FALDH1, FALDH2, FALDH3, and FALDH4 genes, but the present invention is not limited thereto. The FALDH1, FALDH2, FALDH3, and FALDH4 genes may comprise base sequences set forth in SEQ ID NOs: 1 to 4, respectively, but the present invention is not limited thereto.

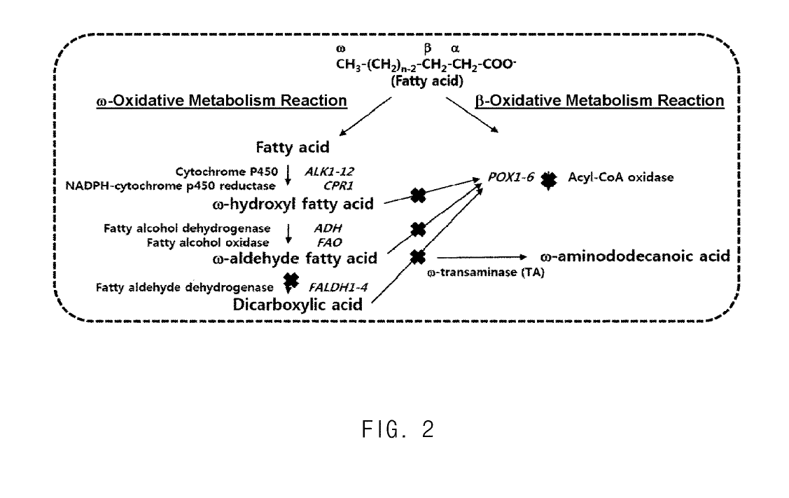

According to another embodiment of the present invention, the β-oxidative metabolism pathway-related genes are preferably deleted from all homologous genes present in the corresponding microorganism, but a recombinant microorganism from which some of these genes are deleted may also be applied to the present invention, when necessary. The β-oxidative metabolism pathway-related genes preferably includes an acyl-CoA oxidase gene, and the acyl-CoA oxidase gene may be selected from the group consisting of ACO1, ACO2, ACO3, ACO4, ACO5, and ACO6 genes, but the present invention is not limited thereto (see FIG. 2). According to other preferred embodiments of the present invention, the ACO1, ACO2, ACO3, ACO4, ACO5, and ACO6 genes may comprise base sequences set forth in SEQ ID NOs: 5 to 10, respectively, but the present invention is not limited thereto.

According to another embodiment of the present invention, the ω-transaminase gene may comprise a base sequence set forth in SEQ ID NO: 11, but the present invention is not limited thereto.

In the present invention, the recombinant microorganism from which the fatty aldehyde dehydrogenase gene and the β-oxidative metabolism pathway-related genes are deleted and into which the ω-transaminase gene is also introduced may be prepared using conventional genetic recombinant technology known in the related art. In the present invention, the term “deletion” is used as a meaning generally encompassing a physical deletion of part or all of the corresponding gene, and also encompassing a situation in which a protein is not expressed from mRNA transcribed from the corresponding gene and a situation in which a protein expressed from the corresponding gene does not function. Also, the term “introduction” is used as a meaning generally encompassing all situations in which a gene is inserted into the genome of a microorganism, or a gene is expressed without insertion of the corresponding gene into the genome of the microorganism. Examples of the genetic recombinant technology that may be used herein may include methods such as transformation, transduction, transfection, microinjection, electroporation, and the like, but the present invention is not limited thereto.

In the present invention, any microorganisms having both ω-oxidative and β-oxidative metabolism processes may be used without limitation. For example, eukaryotes including a yeast and prokaryotes including Escherichia coli may be used. According to an embodiment of the present invention, the yeast is preferably used as the microorganism. In this case, yeasts such as Yarrowia sp., Saccharomyces sp., Pichia sp., Candida sp., and the like may be used as the yeast without limitation. Among theses, Yarrowia lipolytica, Candida tropicalis, Candida infanticola, Saccharomyces cerevisiae, Pichia alcoholophia, or Candida mycoderma is preferably used. Yarrowia lipolytica is more preferably used.

As described above, in the case of the microorganism from which the fatty aldehyde dehydrogenase gene and the β-oxidative metabolism pathway-related gene are deleted and into which the ω-transaminase gene is also introduced, when a fatty acid is supplied as the substrate, an opposite terminal of a carboxyl group is oxidized by an action of cytochrome P450 and an NADPH-cytochrome P450 reductase to form an alcohol. Then, a hydroxyl group of the alcohol is oxidized by an action of a fatty alcohol dehydrogenase and a fatty alcohol oxidase to form an aldehyde. However, because the fatty aldehyde dehydrogenase is deletion, no further oxidation occurs anymore. Also, the fatty acid aldehyde thus formed is aminated by an action of an ω-transaminase to form an aminocarboxylic acid.

Also, the present invention provides a method for producing a medium chain aminocarboxylic acid, which comprises:

(1) preparing a recombinant microorganism from which a fatty aldehyde dehydrogenase gene in an ω-oxidative metabolism pathway and β-oxidative metabolism pathway-related genes are deleted and into which an ω-transaminase gene is also introduced; and

(2) treating the recombinant microorganism with a substrate to culture the recombinant microorganism.

In the present invention, the recombinant microorganism, from which the fatty aldehyde dehydrogenase gene in the ω-oxidative metabolism pathway and the β-oxidative metabolism pathway-related genes are deleted and into which the ω-transaminase gene is also introduced, may be used to produce a medium chain aminocarboxylic acid with high yield by preventing additional oxidation and β-oxidative metabolism of fatty acid aldehydes and introducing an amine group to the medium chain aldehyde fatty acid as well. The fatty aldehyde dehydrogenase gene and the β-oxidative metabolism pathway-related genes are preferably deleted from all homologous genes present in the corresponding microorganism, but a recombinant microorganism from which some of these genes are deleted may also be applied to the present invention, when necessary.

In the present invention, any microorganisms having both ω-oxidative and β-oxidative metabolism processes may be used without limitation. For example, eukaryotes including a yeast and prokaryotes including Escherichia coli may be used. According to an embodiment of the present invention, the yeast is preferably used as the microorganism. In this case, yeasts such as Yarrowia sp., Saccharomyces sp., Pichia sp., Candida sp., and the like may be used as the yeast without limitation. Among theses, Yarrowia lipolytica, Candida tropicalis, Candida infanticola, Saccharomyces cerevisiae, Pichia alcoholophia, or Candida mycoderma is preferably used. Yarrowia lipolytica is more preferably used.

In the present invention, the recombinant microorganism from which the fatty aldehyde dehydrogenase gene and the β-oxidative metabolism pathway-related genes are deleted and into which the ω-transaminase gene is also introduced may be prepared using conventional genetic recombinant technology known in the related art. In the present invention, the term “deletion” is used as a meaning generally encompassing a physical deletion of part or all of the corresponding gene, and also encompassing a situation in which a protein is not expressed from mRNA transcribed from the corresponding gene and a situation in which a protein expressed from the corresponding gene does not function. Also, the term “introduction” is used as a meaning generally encompassing all situations in which a gene is inserted into the genome of a microorganism, or a gene is expressed without insertion of the corresponding gene into the genome of the microorganism.

In the present invention, the “medium chain aminocarboxylic acid” is used as a meaning encompassing all medium chain aminocarboxylic acids having 5 to 30 carbon atoms, preferably 8 to 16 carbon atoms. According to preferred embodiments of the present invention, the medium chain aminocarboxylic acid is preferably 12-aminododecanoic acid having 12 carbon atoms, but the present invention is not limited thereto.

In the present invention, the substrate of step (2) may be a fatty acid, but the present invention is not limited thereto. According to an embodiment of the present invention, a fatty acid having 5 to 30 carbon atoms, preferably 8 to 16 carbon atoms, and more preferably dodecanoic acid having 12 carbon atoms may be used as the fatty acid, but the present invention is not limited thereto.

### MODE FOR INVENTION

Hereinafter, the present invention will be described in further detail with reference to examples thereof.

However, it should be understood that the following examples are just preferred examples for the purpose of illustration only and is not intended to limit or define the scope of the invention.

### Example 1: Construction of Knock-Out Cassette

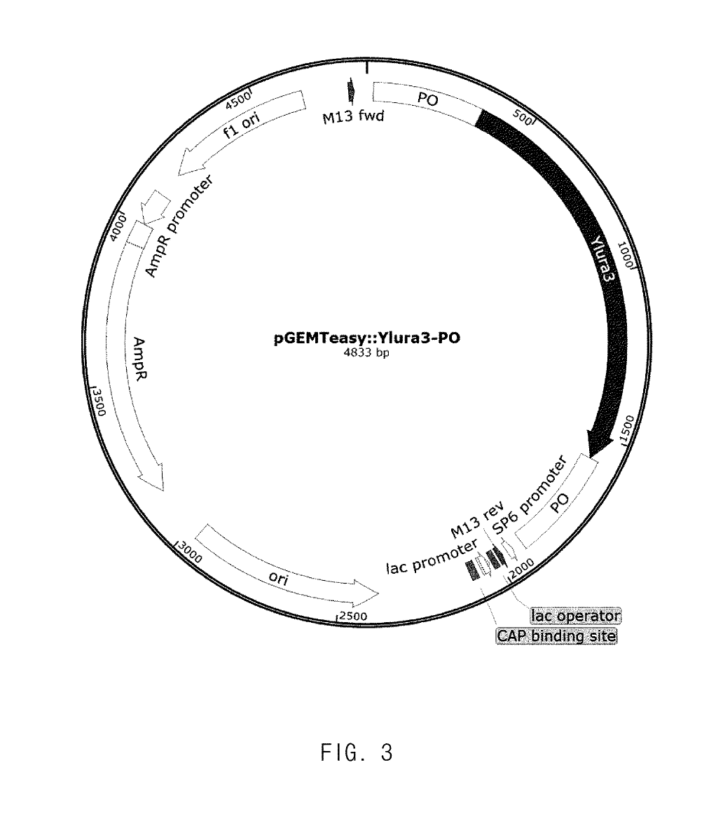

A vector containing an ura3 gene to be used as a selective marker for gene knockout to modify a strain, and a pop-out region for deleting the ura3 gene after insertion of a knock-out cassette was constructed (FIG. 3). A Yarrowia-derived gene was used as the ura3 gene, and the pop-out region used to modify a strain had a total of four sequences, and was referenced from two genes. Here, a Bacillus-derived glutamate-producing gene was used as one of the genes, and a gene associated with a Salmonella- or cloning vector pHUKH-derived His operon was used as the other one. The primers and sequences thereof used to construct the pop-out vectors are listed in the following Table 1.

TABLE 1
Pop-out Vectors

| | | | SEQ ID |
|---|---|---|---|
| Names | | Base Sequences | NOs |
| HisG1 | BglII F | aattgggcccagatctcagaccggttcagacaggat | 13 |
| | EcoRI R | tctctgggcggaattcggaggtgcggatatgaggta | 14 |
| | NotI F | tgTTTCTCGgcggccgccagaccggttcagacaggat | 15 |
| | BamHI R | TCCAACGCGTGGATCCggaggtgcggatatgaggta | 16 |
| HisG2 | BglII F | aattgggcccagatctaacgctacctcgaccagaaa | 17 |
| | EcoRI R | tctctgggcggaattctcttctcgatcggcagtacc | 18 |
| | NotI F | tgTTTCTCGgcggccgcaacgctacctcgaccagaaa | 19 |
| | BamHI R | TCCAACGCGTGGATCCtatctcgatcggcagtacc | 20 |
| glt2 | BglII F | aattgggcccagatctTCAGAACTTGCGCCGATAAA | 21 |
| | EcoRI R | tctctgggcggaattcCTTTGCCAGCTAGACCATAGAG | 22 |
| | NotI F | tgTTTCTCGgcggccgcTCAGAACTTGCGCCGATAAA | 23 |
| | BamHI R | TCCAACGCGTGGATCCCTTTGCCAGCTAGACCAT | 24 |
| | | AGAG | |
| glt3 | BglII F | aattgggcccagatctATTGGCGGGTTCGTTACTT | 25 |
| | EcoRI R | tctctgggeggaattcCCTGGAAGAAGGCCGTATTATC | 26 |
| | NotI F | tgTTTCTCGgcggccgcATTGGCGGGTTCGTTACTT | 27 |
| | BamHI R | TCCAACGCGTGGATCCCCTGGAAGAAGGCCGTAT | 28 |
| | | TATC | |

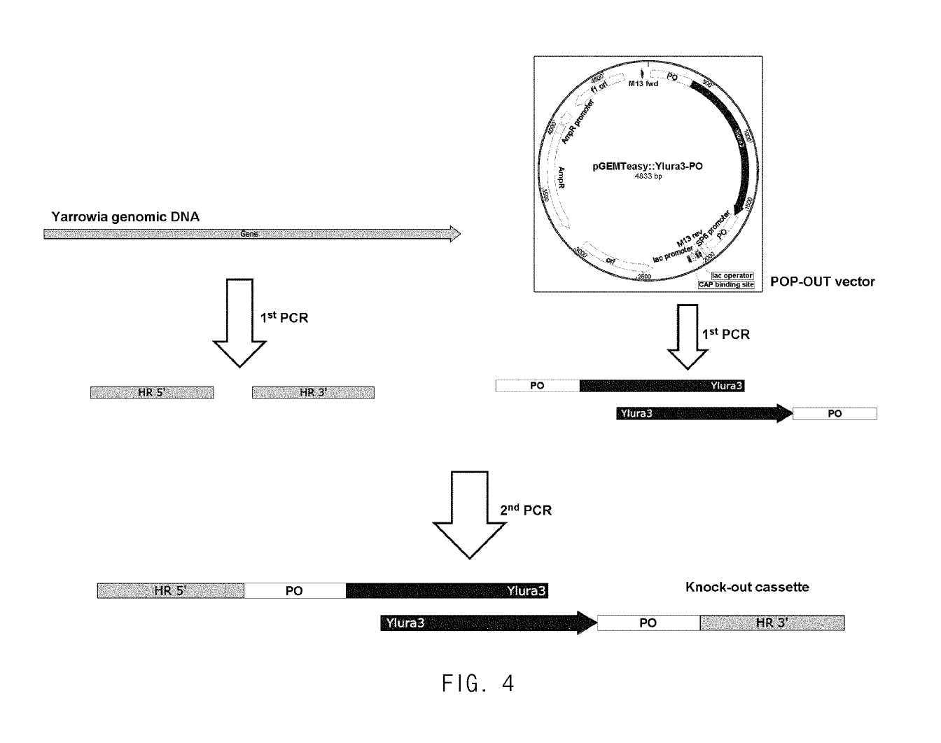

A knock-out cassette was constructed as shown in FIG. 4. First, PCR of a homologous region (HR) to be knocked out from the genomic DNA of Yarrowia sp., and PCR of two 5′- and 3′-terminal fragments from a pop-out vector were carried out separately. Thereafter, each of the 5′ HR and 3′ HR was subjected to alignment PCR (2^{nd} PCR) with a PO-ura3 region to construct a knock-out cassette. The primers and sequences thereof used to amplify the respective homologous regions are listed in Table 2.

TABLE 2
Gene Deletions

| | | | SEQ |
|---|---|---|---|
| | | | ID |
| Names | | Base Sequences | NOs |
| ACO1 | F1 | TTCCTCAATGGTGGAGAAGA | 29 |
| | R1 | TCTTTATCCTGTCTGAACCGGTCTG | 30 |
| | | GTACCATAGTCCTTGCCATGC | |
| | F2 | ATCGCTACCTCATATCCGCACCTCC | 31 |
| | | CTTCTGTCCCCCGAGTTTCT | |
| | R2 | AAGAAGGGCTTGAGAGTCG | 32 |
| ACO2 | F1 | CCCAACAACACTGGCAC | 33 |
| | R1 | TCTTTATCCTGTCTGAACCGGTCTG | 34 |
| | | CTCCTCATCGTAGATGGC | |
| | F2 | ATCGCTACCTCATATCCGCACCTCC | 35 |
| | | gacaagacccgacaggc | |
| | R2 | AGACCAGAGTCCTCTTCG | 36 |
| ACO3 | F1 | Accttcacagagccaccca | 37 |
| | R1 | ATGGCTCTCTGGGCGgtgttgggggtgttgatgatg | 38 |
| | F2 | TTGTTGTGTTTCTCGcaaggttctcatcgaggcctg | 39 |
| | R2 | Aggaaaggtcgaagagtgctct | 40 |
| ACO4 | F1 | Actgcgagagcgatctg | 41 |
| | R1 | TCTTTATCCTGTCTGAACCGGTCTG | 42 |
| | | TTCATGAGCATGTAGTTTCG | |
| | F2 | ATCGCTACCTCATATCCGCACCTCC | 43 |
| | | gaggacgacaaagccggag | |
| | R2 | AGAGCAGAGTCCTCCTCAA | 44 |
| ACO5 | F1 | AACTTCCTCACAGGCAGCGAGC | 45 |
| | R1 | ATGGCTCTCTGGGCG | 46 |
| | | GAGTAGAGAGTGGGAGTTGAGGTC | |
| | F2 | ttgttgtgtttctcg | 47 |
| | | ccccgtcaaggacgctgag | |
| | R2 | ACAGTAAGGTGGGGCTTGACTC | 48 |
| ACO6 | F1 | AGTCCCTCAACACGTTTACCG | 49 |
| | R1 | TCTTTATCCTGTCTGAACCGGTCTG | 50 |
| | | CCATTTAGTGGCAGCAACGTT | |
| | F2 | ATCGCTACCTCATATCCGCACCTCC | 51 |
| | | GAGCTCTGATCAACCGAACC | |
| | R2 | AGGAAGGGTCTAATGACAGA | 52 |
| FALDH1 | F1 | AATCACTCCTCCTACGC | 53 |
| | R1 | TCTTTATCCTGTCTGAACCGGTCTG | 54 |
| | | TGGTCTCGGGGACACCTC | |
| | F2 | ATCGCTACCTCATATCCGCACCTCC | 55 |
| | | CCATCATCAAGCCCCGAA | |
| | R2 | ACCGACATAATCTGAGCAAT | 56 |
| FALDH2 | F1 | Accactaggtgagatcgag | 57 |
| | R1 | TCTTTATCCTGTCTGAACCGGTCTG | 58 |
| | | CTCCGACACTACCGGAACGC | |
| | F2 | ATCGCTACCTCATATCCGCACCTCC | 59 |
| | | CTTGCTCCCACAGTTGTT | |
| | R2 | GATCACCCAGAACCATAGC | 60 |
| FALDH3 | F1 | GTGACCCCCACCACGTCAC | 61 |
| | R1 | TCTTTATCCTGTCTGAACCGGTCTG | 62 |
| | | TTCTGACATTTTCAGCGCCAC | |
| | F2 | ATCGCTACCTCATATCCGCACCTCC | 63 |
| | | CCATTACGAGCGTTTGACGG | |
| | R2 | CAGGGCTGGGGACCACC | 64 |
| FALDH4 | F1 | TACCGACTGGACCAGATTC | 65 |
| | R1 | TCTTTATCCTGTCTGAACCGGTCTG | 66 |
| | | CGGCAGTGGCAATGATCTTAC | |
| | F2 | ATCGCTACCTCATATCCGCACCTCC | 67 |
| | | GACTCGATTCATCGCTCCTAC | |
| | R2 | CAAATCTTTCGGAAGATTCGG | 68 |

The primers used to PCR-amplify the pop-out region and ura3 as two fragments are listed in Table 3.

TABLE 3
Pop-out Cassettes

| | | | SEQ |
|---|---|---|---|
| | | | ID |
| Names | | Base Sequences | NOs |
| HISG1 | F | cagaccggttcagacaggat | 69 |
| | R | ggaggtgcggatatgaggta | 70 |
| HISG2 | F | aacgctacctcgaccagaaa | 71 |
| | R | tcttctcgatcggcagtacc | 72 |
| glt2 | F | TCAGAACTTGCGCCGATAAA | 73 |
| | R | CTTTGCCAGCTAGACCATAGAG | 74 |
| glt3 | F | ATTGGCGGGTTCGTTACTT | 75 |
| | R | CCTGGAAGAAGGCCGTATTATC | 76 |
| Bipartite | Ulura3 cs 2B | Atgccctcctacgaagctcgagc | 77 |
| | Ylura3F | Ctcccaacgagaagctggcc | 78 |

### Example 2: Construction of Transduction Vector

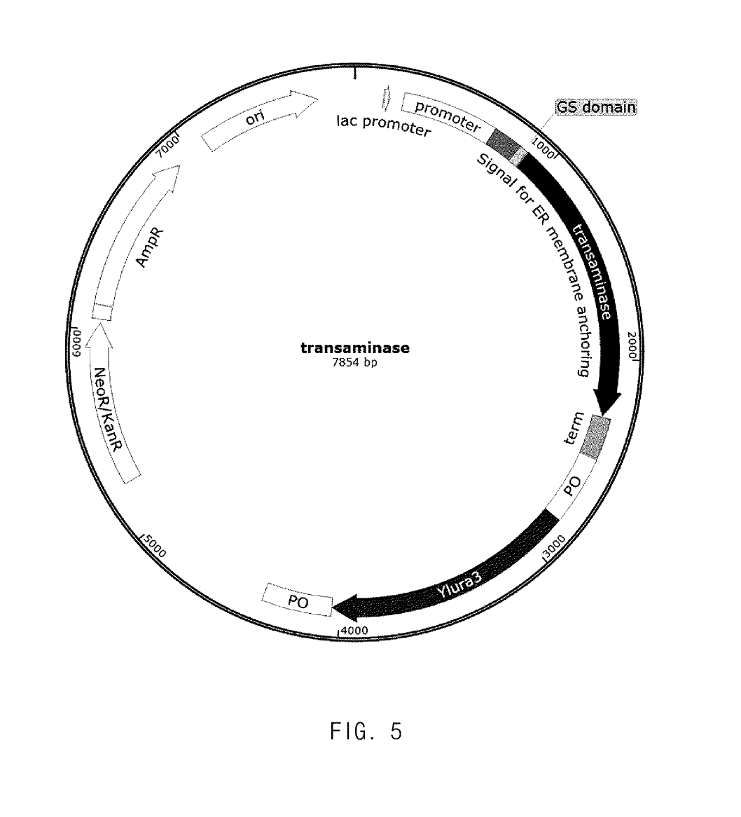

To insert an ω-transaminase into a Yarrowia strain, a vector as shown in FIG. 5 was constructed. The primers used for this purpose are listed in Table 4.

TABLE 4
Transaminase Vectors

| | | SEQ ID |
|---|---|---|
| Names | Base Sequences | NOs |
| EXP 1-F | ccaagcttggtaccgagctcaGagtttggcgcccgttttttc | 79 |
| EXP1-R | CGTTGTTTTTGCATATGTGCTGTAGATATGTCTTGTGTG | 80 |
| | TAA | |
| TEF-F | ccaagcttggtaccgagctcaaactttggcaaagaggctgca | 81 |
| TEF-R | CGTTGTTTTTGCATATGTTTGAATGATTCTTATACTCAG | 82 |
| | AAG | |
| ALK1-F | ccaagcttggtaccgagctcagatctgtgcgcctctacagaccc | 83 |
| ALK1-R | CGTTGTTTTTGCATATGagtgcaggagtattctggggagga | 84 |
| XPR2t-F2 | gtcgacgcaattaacagatagtttgccg | 85 |
| XPR2t-R3 | ctcgagggatcccggaaaacaaaacacgacag | 86 |
| TA-F | CATATGCAAAAACAACGTACTACCTCCC | 87 |
| TA-R | gtcgacTTAGGCCAAACCACGGGCTTTC | 88 |
| ATATG2-ER-F | actcctgcactCATatgtccaacgccctcaacctg | 89 |
| XTATG2-ER-F | ccaatccaacacatatgtccaacgccctcaacctg | 90 |
| ER-R-1 | CGTTGTTTTTGCATAGAACCGCCACCGCCGCTACCGC | 91 |
| | CACCGCCCGAACCGCCACCGCCgaatcgtgaaatatccttgggct | |
| ER-R-2 | CGTTGTTTTTGCATatgAGAACCGCCACCGCCGCTACC | 92 |
| | GCCACCGCCCGAACCGCCACCGCCgaatcgtgaaatatccttgg | |
| | gct | |
| ETATG2-ER-1 | tgattacgccaagcttGagtttggcgcccgttttttc | 93 |
| ETATG2-ER-2 | acaggttgagggcgttggacatATGTGCTGTAGATATGTCTTGTGT | 94 |
| | GTAA | |
| TTATG2-ER-1 | tgattacgccaagcttaaactttggcaaagaggctg | 95 |
| TTATG2-ER-2 | acaggttgagggcgttggacatATGtttgaatgattcttatactcagaag | 96 |
| ER-F | atgtccaacgccctcaacctg | 97 |
| ER-R-3 | CGTTGTTTTTGCATAGAACCGCCACCGCCGCTAC | 98 |

The transaminase cassettes were constructed in the same manner as in FIG. 4, except that, when two fragments of PCR products were obtained from the vector, the genes spanning from a promoter to ura3 were amplified to construct the cassettes. The primers used to construct the cassettes are listed in the following Table 5.

TABLE 5
Transaminase Cassettes

| | | SEQ |
|---|---|---|
| Names | Base Sequences | ID NOs |
| TA-FALDH4-F1 | TACCGACTGGACCAGATTC | 99 |
| TA-FALDH4-R1 | CGGCAGTGGCAATGATCTTAC | 100 |
| TA-FALDH4-F2 | ctcctctatggtctagctggcaaagACTCGATTCATCGCTCCTAC | 101 |
| TA-FALDH4-R2 | CAAATCTTTCGGAAGATTCGG | 102 |
| ATATG2-F | gtcggtaagatcattgccactgccgagatctgtgcgcctctacagac | 103 |
| ETATG2-F | gtcggtaagatcattgccactgccgGagtttggcgcccgttttttc | 104 |
| TTATG2-F | gtcggtaagatcattgccactgccgaaactttggcaaagaggctgc | 105 |
| XTATG2-F | gtcggtaagatcattgccactgccgacgcgtggagagtttgggtt | 106 |

The gene sequences used to modify the recombinant microorganism strain according to the present invention are listed in the sequence listing, and summarized in Table 6.

TABLE 6

| Genes | SEQ ID NOs | Genes | SEQ ID NOs |
|---|---|---|---|
| FALDH1 | 1 | ACO3 | 7 |
| FALDH2 | 2 | ACO4 | 8 |
| FALDH3 | 3 | ACO5 | 9 |
| FALDH4 | 4 | ACO6 | 10 |
| ACO1 | 5 | ω-transaminase | 11 |
| ACO2 | 6 | Ura3 | 12 |

### Example 3: Preparation of Recombinant Microorganism Strain

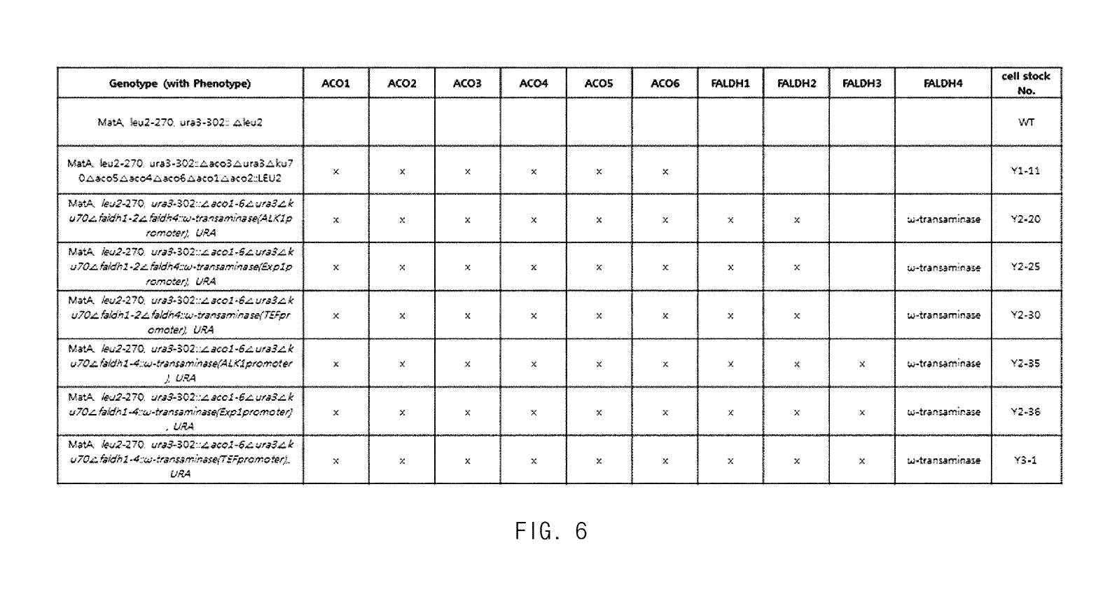

The knock-out cassette constructed in Example 1 and the transduction vector constructed in Example 2 were used to prepare a total of eight knock-out strains from which some of all of a fatty aldehyde dehydrogenase gene in an ω-oxidative metabolism pathway present in a wild-type Yarrowia strain and β-oxidative metabolism pathway-related genes were deleted and into which an ω-transaminase gene was also introduced (FIG. 6). Specifically, a strain in which a gene was to be knocked out or be introduced was plated on an YPD plate, and cultured at 30° C. for 16 to 24 hours. The cultured cells were scraped with a loop, put into 100 μL of a one-step buffer (45% PEG4000, 100 mM DTT, 0.1 L of LiAc, 25 μg of single-strand carrier DNA), and vortexed. Thereafter, the knock-out cassette and the transduction vector (1 ng or more) were added thereto, and the resulting mixture was vortexed again, and then cultured at 39° C. for an hour. The cultured sample was loaded onto a selective medium (6.7 g/L of YNB without amino acids, and 20 g/L of glucose), and then cultured at 30° C. for 48 hours to screen the strains into which the constructed cassette was inserted. To check whether the cassettes were correctly inserted onto the genomes of the screened strains, PCR was then performed using the primers included in the gene deletions listed in Table 2.

To insert another cassette, a pop-out process was performed on the strain into which the cassette was inserted. The strain screened from the selective medium was inoculated in 2 mL of an YPD medium, and cultured at 30° C. for 16 hours, and 200 μL of the culture broth was then spread on a 5′ FOA medium (6.7 g/L of YNB without amino acids, 20 g/L of glucose, 0.8 g/L of 5′ FOA, 0.1 g/L of uracil, and 0.1 g/L of uridine), and then cultured at 30° C. for 48 hours. The strains grown on the 5′ FOA medium were picked, and spread on an YPD plate and a UD plate to screen the strains grown on the YPD plate. Also, a PCR process was again performed using the primers listed in Table 2 to check whether the ura3 gene was deleted from the strains. A knock-out process was performed on other genes of the Ura3-free strains.

### Example 4: Culturing of Recombinant Microorganism Strain

A day earlier, the strain to be cultured and tested was inoculated in 2 mL of an YPD medium (Bacto Laboratories, 10 g/L of Yeast extract, 20 g/L of peptone, and 20 g/L of glucose), and grown at 30° C. and 200 rpm for a day. 2 mL of a growth medium (pH 6.0) having the compositions listed in Table 7 was put into a 24-well plate, and a pre-cultured culture broth was inoculated at 1%. Thereafter, the strains were cultured at 30° C. and 450 rpm for a day in a plate stirrer. The strains cultured for a day were inoculated at a volume of 900 μL in a new plate containing 900 μL of a conversion medium (pH 7.6) listed in Table 8, and 200 μL of a substrate was added thereto at the same time. The resulting mixture was cultured at 30° C. and 450 rpm for a day. In this case, 10 g/L of dodecanoic acid dissolved in DMSO was used as the substrate.

TABLE 7
Growth Medium (pH 6.0)

| Components | Concentration (g/L) |
|---|---|
| Glucose | 50 |
| YNB w/o amino acid | 6.7 |
| Yeast extract | 10 |
| (NH₄)₂SO4 | 5 |
| Uracil | 0.05 |

0.1M phosphate buffer
Preparation of 0.1M potassium phosphate buffer at 25° C.

| | Volume (mL) of | Volume (mL) of |
|---|---|---|
| pH | 1M K₂HPO₄ | 1M KH₂PO₄ |
| 6.0 | 13.2 | 86.8 |

Conversion Medium (pH 7.6)

| Components | Concentration (g/L) |
|---|---|
| Glucose | 30 |
| YNB w/o amino acid | 6.7 |
| Yeast extract | 3 |
| (NH₄)₂SO4 | 15 |
| Uracil | 0.05 |
| L-alanine | 10 |

0.1M phosphate buffer
Preparation of 0.1M potassium phosphate buffer at 25° C.

| | Volume (mL) of | Volume (mL) of |
|---|---|---|
| pH | 1M K₂HPO₄ | 1M KH₂PO₄ |
| 7.6 | 86.6 | 13.4 |

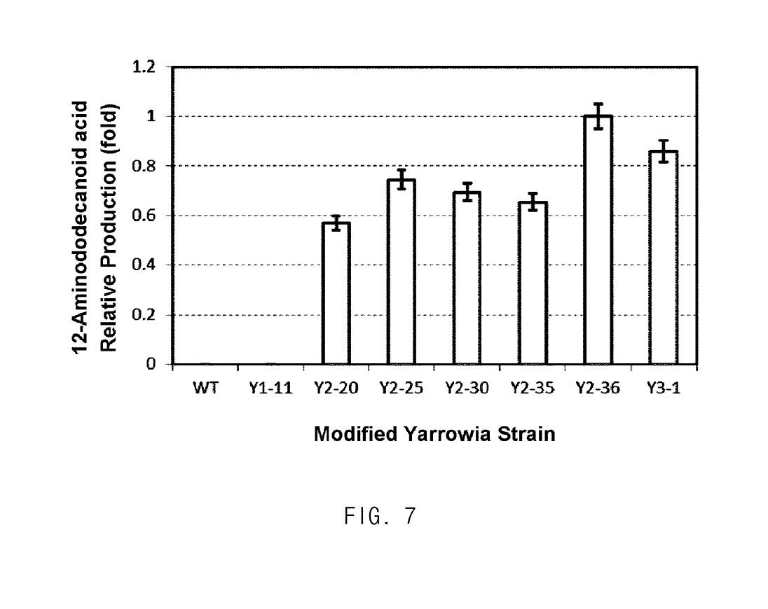

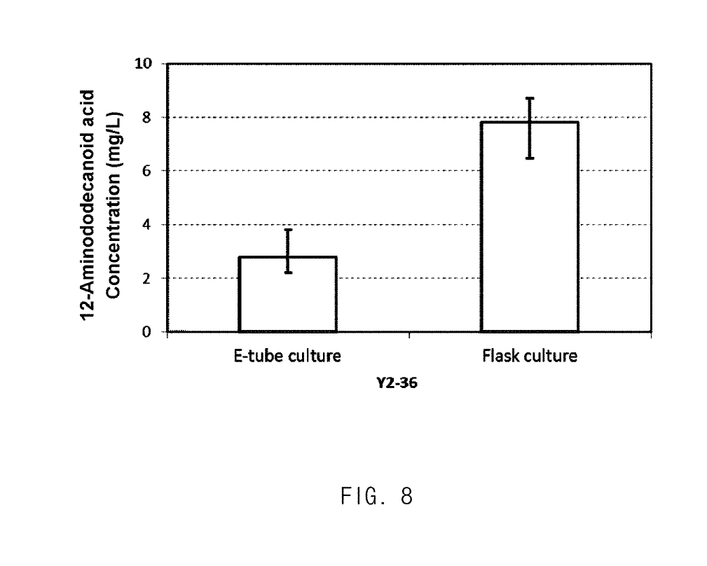

As a result, it was revealed that the Y1-11 strain in which only the β-oxidative metabolism pathway-related genes were knocked out did not produce 12-aminododecanoic acid from dodecanoic acid serving as the substrate, but all the Y2-20, Y-2-25, Y2-30, Y2-35, Y2-36 and Y3-1 strains in which the fatty aldehyde dehydrogenase gene were further knocked out and into which the ω-transaminase was introduced exhibited an excellent ability to synthesize 12-aminododecanoic acid (FIG. 7). Also, it was revealed that the Y2-36 strain exhibited an ability to synthesize approximately 8 mg/L of 12-aminododecanoic acid when cultured in the flask (FIG. 8). In the following experiment, a sample analysis test was performed using the Y2-36 strain.

### Example 5: Sample Analysis

100 μL of 6 N sulfuric acid was added to 500 μL of a culture broth of the Y2-36 strain, which had been proven to have the most excellent ability to synthesize 12-aminododecanoic acid in Example 4, and 500 μL of methanol containing 10% toluene and 2.2% hydrochloric acid was added thereto to perform a methylation reaction. Thereafter, a methylation reaction was performed at 100° C. for an hour. 100 μL of 10 N sodium hydroxide and 500 μL of diethyl ether were added to the reaction solution, and the resulting mixture was thoroughly vortexed, and then centrifuged at 12,000 rpm for 2 minutes. Then, a GC/MS assay was performed under the following analytical conditions to separate only a solvent layer.

Analytical Conditions

\textcircled{1} Equipment: Agilent 5975 MSD

\textcircled{2} Column: HP-5MS

\textcircled{3} Temperature: Oven (150° C. to 230° C.)

\textcircled{4} Carrier Gas: He

\textcircled{5} Flow Rate: 1 mL/min.

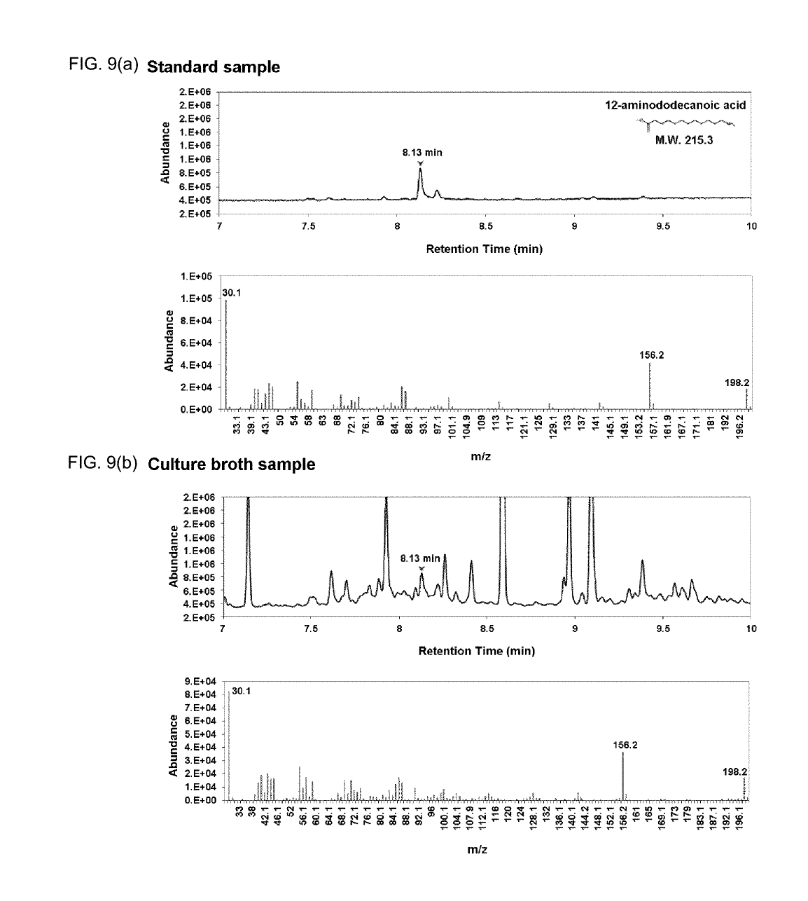

As a result, it was confirmed that the recombinant Y2-36 strain of the present invention was able to synthesize 12-aminododecanoic acid from dodecanoic acid serving as the substrate (FIG. 9).

1. A recombinant microorganism from which a fatty aldehyde dehydrogenase gene in an ω-oxidative metabolism pathway and β-oxidative metabolism pathway-related genes are deleted and into which an ω-transaminase gene is also introduced.

2. The recombinant microorganism of claim 1, wherein the fatty aldehyde dehydrogenase gene and the β-oxidative metabolism pathway-related genes are deleted from all homologous genes present in the microorganism.

3. The recombinant microorganism of claim 1, wherein the fatty aldehyde dehydrogenase gene and the β-oxidative metabolism pathway-related genes are deleted from some of the homologous genes present in the corresponding microorganism.

4. The recombinant microorganism of claim 1, wherein the fatty aldehyde dehydrogenase gene is selected from the group consisting of FALDH1, FALDH2, FALDH3, and FALDH4 genes.

5. The recombinant microorganism of claim 4, wherein each of the FALDH1, FALDH2, FALDH3, and FALDH4 genes comprise base sequences set forth in SEQ ID NOs: 1 to 4.

6. The recombinant microorganism of claim 1, wherein the β-oxidative metabolism pathway-related gene is an acyl-CoA oxidase gene.

7. The recombinant microorganism of claim 6, wherein the acyl-CoA oxidase gene is selected from the group consisting of ACO1, ACO2, ACO3, ACO4, ACO5, and ACO6 genes.

8. The recombinant microorganism of claim 7, wherein each of the ACO1, ACO2, ACO3, ACO4, ACO5, and ACO6 genes comprise base sequences set forth in SEQ ID NOs: 5 to 10, respectively.

9. The recombinant microorganism of claim 1, wherein the ω-transaminase gene comprises a base sequence set forth in SEQ ID NO: 11.

10. The recombinant microorganism of claim 1, wherein the microorganism is a yeast or Escherichia coli.

11. The recombinant microorganism of claim 10, wherein the yeast is selected from the group of the yeast consisting of Yarrowia sp., Saccharomyces sp., Pichia sp., and Candida sp.

12. The recombinant microorganism of claim 11 wherein the yeast of Yarrowia sp. is Yarrowia lipolytica.

13. A method for producing a medium chain aminocarboxylic acid, comprising:
(1) preparing the recombinant microorganism according to claim 1; and
(2) treating the recombinant microorganism with a substrate to culture the recombinant microorganism.

14. The method of claim 13, wherein the substrate is a fatty acid.

15. The method of claim 14, wherein the fatty acid is a fatty acid having 5 to 30 carbon atoms.

16. The method of claim 15, wherein the fatty acid is dodecanoic acid.

17. The method of claim 13, wherein the medium chain aminocarboxylic acid is a medium chain aminocarboxylic acid compound having 5 to 30 carbon atoms.

18. The method of claim 17, wherein the medium chain aminocarboxylic acid is 12-aminododecanoic acid.


SEQUENCE LISTING (106 sequences)

SEQ ID NO: 1 - DNA, 1602 bp, Yarrowia lipolytica
  gene (1)..(1602): FALDH1 gene
atgtcctgggaaacaatcactcctcctacgccaatcgatacgtttgacagcaacttgcaacgtcttcgagactctttcgagaccggcaagctcgactctgtcgactaccgtctcgagcagctgcgaaccctgtggttcaagttctacgacaacctcgacaacatctacgaggcggtcaccaaggatctccatcgacccaggttcgaaaccgagctcaccgaggtactgtttgttcgagacgagttctccaccgtcatcaagaacctgcgaaagtgggtcaaggaagaaaaggtggagaaccccggaggccccttccagtttgccaacccccgaatccgacccgttcctctgggagtggtgctggtcatcactccctggaactaccccgtcatgctcaacatctcacctgtgattgccgccattgctgccggctgtcccatcgtgctcaagatgtccgagctgtctccccacacttccgctgttcttggccgaatcttcaaggaggccctggaccccggtatcatccaggttgtttacggaggtgtccccgagaccaccgcccttcttacccagcattgggacaagatcatgtacaccggaaacggagccgttggtcgaatcatcgcccaggccgcggtcaagaacctgactcctctagctcttgagcttggtggcaagtcacccgtgttcatcacttccaactgcaagagcgttatgacggccgctcggcgaatcgtgtggggcaagtttgtcaacgccggccagatctgtgtcgctccagactacattctggttgctcccgaaaaggaggccgagctcgtcgcttgtatcaaggaggtgctccaagaacgatacggctccaagagagacgcccaccaccccgatctgtcccatatcatttccaagccccattggaagcgtattcacaacatgatcgcccagaccaagggagacatccaggtgggtggactcgagaacgccgacgaagaccaaaagttcatccagcccacaatcgtctccaacgttccagatgacgacattctcatgcaggacgagattttcggacccatcatccccatcatcaagccccgaaccctcggccagcaggttgattacgtcacaagaaaccatgacacccccctggccatgtacatcttctctgacgaccccaaggaggtggactggctacagacccgaatccgagctggttctgtaaacatcaacgaggtcattgagcaggtcggactggcctctctgcctctcagtggagttggagcttccggaaccggagcataccatggaaaattctccttcgatgtcttcacccacaagcaggccgttatgggacagcccacctggcccttctttgaatacctcatgtattaccggtaccctccttactccgagtacaagatgaaggtgctccgaaccctgttcccaccggttctgattcctcgaaccggccgacccgacgctactgttcttcagcgagttctcggcaacaagctgctttggatcattattgccgcccttgttgcgtacgccaaacgaaatgagctgctcatcaccattgctcagattatgtcggtgtttattaagtag

SEQ ID NO: 2 - DNA, 1566 bp, Yarrowia lipolytica
  gene (1)..(1566): FALDH2 gene
atgtcagagttcgattgggagtcaattttgccggcaacaccactaggtgagatcgagaaggatattcaaaccctacgacagggcttcaggtccggaaagacgctggatttgaacttcaggcttgaccagattcgtaagcttttctatgctctctatgataatgtcgatgcgatcaaagaagcaattcataaggatctcggacgtccggtcttcgagactgaactttgcgagatctcctttcagtggggtgaattcaataatgtcgtttctaacttgaagaaatgggcagctgatgagacggtgaagggaaccaccattcaatacactctcacccggccaaagattagaaagcgtccacttggtaccgtccttatcatatctccttggaactacccatttgttctgaccatctctcccctgcttgctgctctagcggcaggaaatacggtggccctaaagttctccgaaatgtgcccacatacatcgcttttgctgggaaagttgtgcacagaggcacttgataaagaaattttcaaggcatttcagggaggcgttccggtagtgtcggagattctcaagtacaagttcgacaaaatcatgtacactggaaatcatcgagttggcaagatcatcttggacgcagctaacaaatacctcacccccgttattttggagcttggaggcaaatcaccagtcttcgtgactaagaattgccaaaacgtatctcttgctgccaagcgtgctctgtggggtaaactggtcaacgctggacaaacatgcgttgcccccgattacatcatcgtcgagcctgaggtcgaacaggagtttatcaaagcttgccagtactgggttgagaagttctaccgaggtggagttgactctgatcataaggacttcactcatattgcaacacctggacattggagacgattgacatccatgcttgcccagacagagggaaatatcatcacaggcggaaattcggacgagaaatcacggtttcttgctcccacagttgttgcgaaagttcctgatggtgattctttgatgaatgatgagatctttggccctatcctgcccatcctgacagccagatccgttgacgaaggtattcgctatgttcatgagaatcacgacactcccctggccatgtatgtctttactgataatgcatcagaaggagagtatatccaatctcaaatcaactcaggtggcctgatattcaatgatagtcttgttcacgttggctgcgtgcaggcgccttttggtggtgtcggccaatccggctatgggtcttatcacggcgaagattccttcttggctttttcacacaggcagactttcatgaagcagccccatttcatcgaacgaccaatggcgatcagatatgccccctacactagtcgaaaacaaaaggctgtccagggtagtctagctgctccatcttttcctcgaacaggaaaggttgaccgctccctgttggagcggatatttggtaagctatggttctgggtgatcgttttagggctaggagcagccagtttgaagtcaggaattttcttatga

SEQ ID NO: 3 - DNA, 1590 bp, Yarrowia lipolytica
  gene (1)..(1590): FALDH3 gene
atgactaccactgccacagagacccccacgacaaacgtgacccccaccacgtcactgcccaaggagaccgcctccccaggagggaccgcttctgtcaacacgtcattcgactgggagagcatctgcggcaagacgccgttggaggagatcgagtcggacatttcgcgtctcaaaaagaccttccgatcgggcaaaactctggatctggactaccgactcgaccagatccgaaacctggcgtatgcgatccgcgataacgaaaacaagatccgcgacgccatcaaggcggacctgaaacgacctgacttcgaaaccatggcggccgagttctcggtccagatgggcgaattcaactacgtggtcaaaaacctgccgaaatgggtcaaggacgaaaaagtcaagggaaccagcatggcgtactggaactcgtcgccaaagatccggaaacggcccctgggctccgtgcttgtcatcacgccctggaactacccactgattctggccgtgtcgcctgttctgggcgccattgccgcaggcaacaccgtggcgctgaaaatgtcagaaatgtcacccaacgcgtcaaaggtgattggcgacattatgacagctgccctggacccccagctctttcaatgcttcttcggaggagtccccgaaaccaccgagatcctcaaacacagatgggacaagatcatgtacaccggaaacggcaaagtgggccgaatcatctgtgaggctgccaacaagtacttgacacctgtggagctcgaactcggaggaaagtcgcctgttttcgtcaccaaacactgctccaacctggaaatggccgcccgccgaatcatctggggcaaattcgtcaacggaggacaaacctgcgtggctccagactacgttctggtgtgtcccgaggtccacgacaaatttgtggctgcctgtcaaaaggtgctggacaagttctaccctaacaactctgccgagtccgagatggcccatatcgccacccctctccattacgagcgtttgacgggcctgctcaattccacccgaggtaaggtcgttgctggaggcactttcaactcggccacccggttcattgctcctacgattgtcgacggagtggatgccaacgattctctgatgcagggagaactgtttggtcctcttctccccattgtcaaggccatgagcaccgaggctgcctgcaactttgtgcttgagcaccaccccacccccctggcagagtacatcttttcagataacaattctgagattgattacatccgagatcgagtgtcgtctggaggtctcgtgatcaacgacactctgatccacgtgggatgcgtacaggcgccctttggaggtgtcggagacagtggaaatggaggataccatggcaagcacactttcgatttgttcagccattctcagacggtcctcagacaacccggatgggtcgaaatgctgcagaagaaacggtatcctccgtacaacaagagcaacgagaagtttgtccggagaatggtggtccccagccctggttttccccgggagggtgacgtgagaggattttggtcgagactcttcaactag

SEQ ID NO: 4 - DNA, 1560 bp, Yarrowia lipolytica
  gene (1)..(1560): FALDH4 gene
atgtctacctttgattgggaatccattgtgcctgccactcctctcgaccagattcctggcgacatccagcgactgcgaaagggcttccgatccggaaagaccctcgatctcaactaccgactggaccagattcgaaacttgcactacgtcctcagagacaatgtcgaggccatcaaggacgccgtgtacaaggatctcggccgacccaagcacgagactgacctgtgcgaggtgggtttcctgtggggcgagtttaacaacgtggttgccaacctcaagaagtgggccgccgacgaggacgtcaagaccaacctgcagtactccatctcctcccccaagatccgaaagcgacctcttggaaacgtgctcatcatctcgccctggaactacccctttatgctgaccgtgtctcctctcattggagctctggctgccggtaacactgtggctgtcaagttctccgaaatggccccccacacttccaaaattgttggcgacttgtgcaccaaggccctcgaccccgacgtcttccaggccatccagggaggtgtccccgtcgtcaccaagaccctcgagcagaagttcgacaagattatgtacactggtaaccacactgtcggtaagatcattgccactgccgccaacaagtacctgacacccgtcatcctcgagctcggaggtaagtcgcccgtttttgtcaccaagaactgcaagaacatcaagcttgccgctaagcgagccctgtggggtaaggtggtaaacgctggccagacctgtgtggctcccgactacgtgattgtcgagcccgaggtggagcaggagtttatcgacgcctgcaagtactggattaacgagttctacagtggtaagattgaccagtacaaccccgactttgccaagatcgccacccccaaccactggaaccgacttacctccatgttgagcaagtccaagggagagatcattactggaggtaacactgacgagaagactcgattcatcgctcctactgtcgtcgcaaaggtccccgacaatgattccctgatggaggacgagattttcggccctcttctgcccattctcactgcccgatccgtcgaggagggtatcaagtacgtgcacgagaaccacgacacccctcttgccatgtacgtcttcactgacaaggcctctgagggcgactacatccagtcccagatcaactctggtggccttatcttcaatgacactctgatccacgttggatgtgtccaggctccgtttggtggtgtcggcatgtccggttacggtgcttaccatggcgaggactccttcctggccttcacccaccgacaaacctacctcaaccagcccaagcttctggagcctcttcaggacgtgcgatacgccccctacaccaaaaccaagcgaagcatggtcaagaacctgctgctggtcggccccattttcccccgaaccggctccgtataccccaacgtgctgatccgaatcttccgaaagatttggttctgggtccttattgtcgccatcggagctgctggtgccaaggctctgctctag

SEQ ID NO: 5 - DNA, 2034 bp, Yarrowia lipolytica
  gene (1)..(2034): ACO1 gene
atggccaaggagcgaggtaagactcaattcactgtccgagatgtgaccaacttcctcaatggtggagaagaagagacccagattgtcgagaagatcatgagcagtattgaacgtgatccagtactgtctgtcactgctgactacgactgcaaccttcagcaggcccgaaaacagaccatggagcgggtggctgctctgtcgccttatctggtcaccgatactgagaagctatctctgtggcgtgcgcaactgcatggaatggttgatatgtctactcgtacgcggttgtcgatccacaacaacctgttcattggttccatcaggggatctggtactcctgaacagttcaagtactgggtcaagaagggagcggtggctgttaagcagttctatggatgctttgccatgacagagttgggccatggaagcaacctcaagggactagagacaaccgccacttatgaccaggacagtgaccagttcattatcaacactcctcatattggtgctaccaagtggtggattggcggtgcagcccacacttccacccattgtgtttgtttcgcgaaactgattgtgcatggcaaggactatggtactcgaaactttgtggtacctctccgaaatgtccacgatcacagtctcaaggtcggtgtttcaattggagacattggaaagaagatgggcagagatggtgttgacaatggctggatccagttcaccaatgttcgaatccccagacagaacatgctaatgagatatgccaaggtgtctgatactggagtggtaaccaaacccgctcttgaccaactcacttatggagccctcattcgaggtcgagtgtccatgattgccgactcgttccacgtctccaaacgattcctcacaattgctcttcggtacgcttgtgtccgacgacagtttggaacctctggagacactaaggagaccaagatcatcgactacccttaccaccagcgacgattgctgcctcttctggcctactgctacgctatgaagatgggtgctgatgaggctcagaagacttggattgagaccaccgatcgaattctggctctcaatcccaacgaccccgcccagaagaacgatctggagaaggccgtcaccgacacaaaggagctgtttgctgcgtctgcaggaatgaaggcatttaccacgtggggatgtgccaaaatcattgatgagtgccgacaggcctgtggaggtcatggatactctggatataacggatttggccagggctacgctgactgggttgtccagtgtacctgggaaggagacaacaacgttctgtgtctgtcaatgggccgagggctggttcagtcagctctacagattttggctggaaagcacgtcggtgcttctattcagtacgtaggagacaagtctaaaatctcccagaacggccagggtacccccagagagcaacttctgtcccccgagtttctagtagaagctttcagaacggcttctcgaaacaacattctcagaaccaccgataaataccaagagcttgtcaaaactctcaatcccgaccaggcctttgaggagctgtctcagcagagattccagtgtgctcgaatccacacacgacagcatcttatctcttcattctatgcccgaattgccactgccaaagacgatatcaagccccatctgctgaaactggccaatctgtttgccctctggtcaattgaggaggacactggaatcttcctgcgggagaacatcctcacccctggagacattgacctgatcaacagtcttgtggacgagctctgtgttgcagttcgagatcaggtaattggactcactgatgcctttggtctctctgacttcttcattaacgctcccatcggctcctacgatggtaatgtttacgaaaagtactttgccaaggtcaaccagcaaaaccccgctactaaccctcgtcctccctactacgagtcgactctcaagcccttcttgttccgagaagaggaggacgatgaaatttgcgatctcgatgagtga

SEQ ID NO: 6 - DNA, 2103 bp, Yarrowia lipolytica
  gene (1)..(2103): ACO2 gene
atgaaccccaacaacactggcaccattgaaatcaacggtaaggagtacaacaccttcaccgagccccccgtggccatggctcaggagcgagccaagacctccttccccgtgcgagagatgacctacttcctcgacggtggcgagaagaacaccctcaaaaacgagcagatcatggaggagattgagcgagaccctcttttcaacaacgacaactactacgatctcaacaaggagcagatccgagagctcaccatggagcgagtcgccaagctgtctctgtttgtgcgtgatcagcccgaggacgacatcaagaagcgatttgctctcattggtatcgccgatatgggaacctacacccgacttggtgtccactacggcctcttctttggcgccgtccgaggtaccggaactgccgagcagtttggccactggatctccaagggagccggagacctgcgaaagttctacggatgtttctccatgaccgagctgggccatggctccaacctggctggtctcgagaccaccgccatctacgatgaggagaccgacgagttcatcatcaacacccctcacattgccgccaccaagtggtggattggaggagccgcccacaccgccacccacactgtcgtgttcgcccgactcattgtcaagggcaaggactacggtgtcaagacctttgttgtccagctgcgaaacatcaacgaccacagcctcaaggtcggtatctctattggtgatatcggaaagaagatgggccgagacggtatcgataacggatggatccagttcaccaacgtgcgaatcccccgacagaacctgctcatgaagtacacaaaggtcgaccgagagggtaacgtgacccagcctcctctggctcagcttacctacggttctcttatcactggtcgagtctccatggcctctgattctcaccaggtcggaaagcgattcatcaccattgctctgcgatacgcctgcattcgacgacagttctccaccacccccggccagcccgagaccaagatcatcgactacccctaccatcagcgacgacttctgcctcttctggcctatgtctatgctcttaagatgactgccgatgaggttggagctctcttctcccgaaccatgcttaagatggacgacctcaagcccgacgacaaggccggcctcaatgaggttgtttccgacgtcaaggagctcttctccgtctccgccggtctcaaggccttctccacctgggcttgtgccgacgtcattgacaagacccgacaggcttgcggtggccacggttactctggatacaacggtttcggccaggcctacgccgactgggttgtccagtgcacctgggagggtgacaacaacattctcaccctttctgccggccgagctcttatccagtctgccgttgctctgcgaaagggcgagcctgttggtaacgccgtttcttacctgaagcgatacaaggatctggccaacgctaagctcaatggccgatctctcaccgaccccaaggtcctcgtcgaggcctgggaggttgctgccggtaacatcatcaaccgagccaccgaccagtacgagaagctcattggcgagggtcttaacgccgaccaggcctttgaggttctgtctcagcagcgattccaggccgccaaggtccacacacgacgacacctcattgccgctttcttctcccgaattgacaccgaggctggcgaggccatcaagcagcccctgcttaacctggctctgctgtttgccctgtggtccatcgaagaggactctggtctgttcctgcgagagggcttcctcgagcccaaggatatcgacaccgtcaccgagctcgtcaacaagtactgcaccactgtgcgagaggaggtcattggctacaccgatgccttcaacctgtccgactacttcatcaacgctcctattggatgctacgatggtgacgcttaccgacactacttccagaaggtcaacgagcagaaccctgcccgagacccccgacctccttactacgcctctactctcaagcccttccttttccgagaggaggaggatgatgacatttgcgagcttgatgaggaatag

SEQ ID NO: 7 - DNA, 2103 bp, Yarrowia lipolytica
  gene (1)..(2103): ACO3 gene
atgatctcccccaacctcacagctaacgtcgagattgacggcaagcagtacaacaccttcacagagccacccaaggcgctcgccggcgagcgagccaaggtcaagttccccatcaaggacatgacggagtttctgcacggtggcgaggagaacgtgaccatgatcgagcgactgatgacggagctcgagcgagaccccgtgctcaacgtgtcgggcgactacgacatgcccaaggagcagctgcgagagacggccgtggcgcgaattgcggcgctgtccggccactggaagaaggacacagaaaaggaggcgctgctgcggtcccagctgcacggcattgtggacatgggcacccgaatccgactcggtgtgcacacgggcctgttcatgggcgccatccggggttccggcaccaaggagcagtacgactactgggtgcgaaagggcgccgcggacgtcaagggcttctacggctgctttgctatgaccgagctgggccatggctccaacgtggccggtcttgagaccaccgccacctacatccaggacacggacgagttcatcatcaacacccccaacactggagccaccaagtggtggattggaggagccgcccactcggccacccacaccgcctgctttgctcgtctgcttgtcgacggcaaggactacggcgtcaagatctttgttgtccagctgcgagacgtctcttctcactctctcatgcccggcatcgctctcggcgacattggaaagaagatgggccgagacgccatcgacaacggctggatccagttcaccaatgtgcgaatcccccgacagaacatgctcatgaagtacgccaaggtctcgtctaccggcaaggtgtcgcagcctcctctggcccagctcacctacggcgctctcattggcggccgagtcaccatgattgccgactccttctttgtctcccagcgattcatcaccattgctctgcgatacgcctgtgtgcgacgacagtttggcaccacccccggccagcccgagactaagatcatcgactacccctaccatcagcgacgtctgctgcctcttctggccttcacctacgccatgaagatggccgccgaccagtcccagattcagtacgatcagaccaccgatctgctgcagaccatcgaccctaaggacaagggcgctctgggcaaggccattgtcgacctcaaggagctgtttgcctcttctgctggtctcaaggccttcaccacctggacctgtgccaacatcattgaccagtgccgacaggcctgcggtggccacggctactctggctacaacggctttggccaggcctacgccgactgggttgtccagtgcacctgggagggtgacaacaacgtcctgtgtctgtccatgggccgaggtctcatccagtcgtgtctgggccaccgaaagggtaagcctctgggctcttctgtcggctacctggctaacaagggtcttgagcaggctactctgagcggccgagacctcaaggaccccaaggttctcatcgaggcctgggagaaggtcgccaacggcgccatccagcgggccactgacaaatttgtcgagctcaccaagggcggcctctctcctgaccaggcctttgaggagctgtcgcagcagcgattccagtgtgccaagatccacacccgaaagcacctggtgactgccttctacgagcgaatcaacgcctctgcgaaggccgacgtcaagccttacctcatcaacctcgccaacctcttcactctgtggtccattgaggaggactctggtctcttcctgcgagagggtttcctgcagcccaaggacattgaccaggtgactgagctggtgaaccactactgcaaggaggttcgagaccaggttgccggctacaccgatgcctttggtctgtctgactggttcatcaacgctcccattggaaactacgatggtgacgtttacaagcattactttgccaaggttaaccagcagaaccctgctcagaacccccgacctccttactatgagagcactcttcgacctttcctgttccgagaggatgaggatgacgacatttgcgagctggacgaggaatag

SEQ ID NO: 8 - DNA, 2106 bp, Yarrowia lipolytica
  gene (1)..(2106): ACO4 gene
atgatcaccccaaaccccgctaacgacattgtccatgacggcaagctctacgacaccttcactgagccccccaagctgatggctcaggagcgagctcagctggacttcgaccctagagacatcacctactttctggatggctctaaggaggagaccgagctgctggagtcgctcatgctcatgtacgagcgagaccctctcttcaacaaccagaacgagtacgatgaatcgtttgaaacactgcgagagcgatctgtgaagcgaattttccagctgtccaagtccatcgccatggaccccgagcccatgtctttccgaaagattgggttcctgggtattcttgacatgggaacgtatgctcgactgggagtccactacgcgctcttctgtaactccatccggggccagggaacccccgatcagctcatgtactggctggaccagggagccatggtcatcaagggcttctacggctgttttgccatgaccgaaatgggccatggatctaacctgtcgcgtctggaaaccatcgccactttcgacaaagagaccgacgaatttatcattaacacgccccacgttggagccacaaagtggtggattggtggtgctgctcacactgctactcacacacttgcctttgcccgtcttcaagtagacggaaaggactacggtgtgaaatcgtttgtcgtacctctccgaaacctggacgaccattcgctgcgtcctggaatcgccacaggtgatattggtaagaagatgggtcgagatgccgttgacaacggctggattcagttcaccaacgtccgagtgccccgaaactacatgctcatgaagcataccaaggttcttcgagacggtaccgtcaagcagccgcctttggcccaactgacttacggatctctcatcactggacgagtccagatgaccactgactctcacaatgtgtccaaaaagttcctcaccattgccctgagatacgccaccatccgacgacagttctcgtcaactccaggagagcccgaaacccgactaattgactacctgtaccaccaaagacgactcctgcctcttatggcttactcttacgccatgaaactagctggagatcacgtccgagagctgttctttgcatcccaggagaaggctgagagcctcaaggaggacgacaaagccggagttgagtcttacgtccaggatatcaaggagctcttctctgtttctgctggtctcaaggctgccactacatgggcttgtgctgacatcattgacaaggcccgacaggcgtgtggaggccacggatactctgcctacaacggctttggacaggccttccaggactgggttgtccagtgcacttgggagggtgacaatactgttctgactctatctgccggccgagctctgatccaatctgctctcgtctaccgaaaggagggcaaactaggtaacgccacgaagtacctctctcggtccaaggagcttgccaacgccaagagaaacggacgatccctggaagaccccaagctgctcgtggaggcatgggaggctgtctctgccggtgctatcaacgctgctactgacgcttacgaggagctctccaagcagggagtttctgttgacgagtgctttgagcaggtgtcccaggagcgattccaggctgcccgaatccacactcgacgagctcttatcgaggccttctactcacgaatcgccactgctgatgagaaggtgaagcctcatctgatccctctggccaacctgtttgccctgtggtccattgaggaggactctgctctgttcctggctgagggctactttgagcctgaggatatcattgaggtgacttctcttgtcaacaagtactgcggaattgttcgaaagaacgttattggatacaccgatgccttcaacctgtccgactacttcatcaacgctgccattggacgatacgacggagacgtgtacaagaactactttgagaaggtcaaacagcagtaccctcctgagggtggcaagcctcactactacgaggatgtcatgaagcccttcctgcatcgagagcgaattcccgatgtccccatggagcccgaggatattcagtaa

SEQ ID NO: 9 - DNA, 2100 bp, Yarrowia lipolytica
  gene (1)..(2100): ACO5 gene
atgaacaacaaccccaccaacgtgatccttggaggcaaggagtacgacaccttcaccgagcctccggcccagatggagctggagcgagccaagacacaattcaaggtccgagacgtgaccaacttcctcacaggcagcgagcaggagacactgctgaccgagcgaatcatgcgggagattgagcgagatcccgttctcaacgtcgccggcgactacgacgccgatcttcccaccaagcgacgacaagctgttgagcgaatcggggctctggcccgatacctgcccaaggattccgagaaggaggccattttgcgaggccagctgcatggtattgtggacatgggtacccgaacccgaatcgccgttcactacggtctgtttatgggcgccattcgtggctcaggaaccaaggagcagtacgattactgggtcgccaagggcgccgctactctgcacaaattctatggctgctttgccatgactgagctgggtcacggatctaacgtggccggtctcgagaccaccgccacccttgataaggacaccgacgagttcatcatcaacacccccaactcgggagccacaaagtggtggattggaggagctgcccactctgctacccacacggcttgtcttgcccgactcattgttgatggcaaggactatggtgttaagatcttcattgttcagctgcgagacctcaactcccactctctactcaacggtattgccattggagatatcggcaagaagatgggccgagatgccattgataatggttggatccagttcacagacgtccgaattccccgacagaacatgctcatgcgatacgaccgggtgtctcgagacggcgaggttaccacctccgagcttgcccagctcacctacggagcacttctgtctggccgagtgaccatgattgccgagtctcacctcctgtctgctcggttcctcaccattgctcttcggtacgcctgtatccgtcgacagttcggagctgtgcctgacaagcccgagactaagctcatcgactacccctaccaccaacgacgtctgctgcctcttctggcctacacctacgccatgaagatgggcgccgacgaggcccagcagcagtacaactcctcctttggcgctcttctcaagctcaaccccgtcaaggacgctgagaagtttgctgtcgccactgccgacctcaaggctctgtttgcctcttctgccggaatgaaggccttcaccacctgggctgccgccaagatcattgacgagtgccgacaggcctgtggtggccatggctactccggctacaacggtttcggtcaggcttacgccgactgggtcgtccaatgcacttgggagggtgacaacaacgtgctgtgtctgtccatgggtcgatcgctcatccagtcgtgcattgccatgagaaagaagaagggccatgtcggcaagtcggtcgagtacctgcagcgacgagacgagctgcagaatgcccgagttgacaacaagcctctcactgaccctgctgtgctcatcactgcatgggagaaggttgcctgcgaggccatcaacagagccactgactccttcatcaagctcacccaggagggtctgtctcctgaccaggcctttgaggagctgtctcaacagagatttgagtgtgcgcgaatccacacccgaaagcatctgatcacctcgttctacgctcgaatctccaaggccaaggcccgagtcaagccccaccttactgttcttgccaacctctttgccgtctggtccatcgaggaggactctggtctcttccttcgggagggctgcttcgagcctgccgagatggacgagatcaccgctctggtcgacgagctgtgctgcgaggctcgagagcaggtcattggattcaccgacgccttcaacctgtccgacttcttcattaacgcccccattggccgattcgacggagacgcctacaagcactacatggacgaggtcaaggctgccaacaaccctcgtaacacccatgctccttactacgagaccaagctgcgacccttcctgttccgacccgatgaggacgaggagatttgcgacctggacgagtag

SEQ ID NO: 10 - DNA, 2070 bp, Yarrowia lipolytica
  gene (1)..(2070): ACO6 gene
atgctctctcaacagtccctcaacacgtttaccgagcccccggtcgaaatggcccgggagcgaaaccagacttccttcaacccgcgtctgctgacgtactttctggacggaggcgaaaagaacactctgcttatggaccgactgatgcaagagtacgagcgagaccctgtgtttcgaaacgagggcgactacgatattaccgatgtggcccagtcgcgagagctggccttcaagcgaatcgccaagctcatcgagtatgtgcacaccgacgacgaggagacgtatctgtaccgatgcatgcttctgggccaaatcgatatgggagcctttgcccggtacgccatccaccacggagtctggggcggtgccattcgaggtgcaggaacgcctgagcagtacgaattctgggtcaagaaaggatctctgtcggttaagaagttctatggatccttctccatgaccgagctgggccacggcagtaacttggtgggtctggagaccaccgccaccctggacaagaacgcagacgagttcgtgatcaacactcccaacgttgctgccactaaatggtggatcggaggagccgccgataccgccactcacacagctgtgtttgcacgtctcattgtcgacggagaggaccacggtgtcaagacgtttgtggtgcagctgcgagacgtggagactcacaacctgatgcctggtattgctatcggagactgcggcaagaagatgggacgtcagggaaccgacaacggctggatccagttcacccatgtgcgaattccccgacagaacatgctcatgcgatactgtcacgtggacagcgacggaaatgttaccgagcccatgatggctcagatggcctacggagctcttctggctggccgagtcggaatggccatggacagttatttcacctcgcgaaagttccttaccattgctcttcgatatgccaccattcgacgagcttttgctgccggaggaggtcaggagaccaagctgatcgactacccttaccaccagcgacgtctgctccccctcatggcccagacatatgccatcaagtgcaccgccgataaggtcagagatcagttcgtcaaggtcaccgacatgctcctaaacctcgatgtttctgaccaagaggccgtgcccaaggccattgccgaggctaaggagctcttctctgtttctgctggtgtcaaggctaccacaacttgggcttgcgcacacaccattgaccagtgcagacaggcgtgtggaggccacggatactctgcttacaacggttttggacgtgcttactccgattgggtgatccagtgcacctgggagggagacaataacattctgtgtctgtcagctggcagagctctggtccagtctaaccgagctgtccgggctggcaagcccattggaggtcctaccgcctacctggctgctcccgctggttcccccaagctcgctggtcgaaacttgtacgaccccaaggtcatgattggggcctgggagactgtttcccgagctctgatcaaccgaaccaccgatgagtttgaggtgctggccaagaagggtctgtctactgcccaggcctacgaggagctgtcccagcaacgattcctgtgtactcgaatccacacccgtctgtacatggtcaagaacttctacgagcgaattgccgaggagggcaccgagttcaccaaggagcctcttaccagacttgccaacctgtacgccttctggtccgtcgaagaggaggctggaatcttcctccgagagggctacatcactccccaggagctcaagtacatcagtgccgagatccgaaagcagctcttggaggtgcgaaaggacgtcattggctacaccgatgccttcaacgtgcctgattttttcctcaactctgccattggacgagctgacggagatgtctacaagaactacttcaaggtggtcaacactcagaaccctccccaagaccctcgacctccttattacgagtctgtcattagacccttcctgttccgaaaggacgaggatgaggaaatttgctctcttgaggatgagtag

SEQ ID NO: 11 - DNA, 1380 bp, Chromobacterium violaceum DSM30191
  gene (1)..(1380): transaminase gene
atgcaaaaacaacgtactacctcccaatggagagaattggatgcagcccatcatttgcatccatttaccgacaccgcatccttgaatcaagctggcgcacgtgtgatgacccgtggcgagggcgtttacctgtgggattctgagggtaacaaaatcattgatggtatggccggtttgtggtgtgtgaacgtgggttatggtcgtaaagacttcgcagaagcagcccgtcgtcagatggaagaattgcctttctacaacactttcttcaaaactacccacccagctgtggtcgagttgtcttccttgttggccgaagttaccccagctggtttcgaccgcgttttctacaccaattcgggctccgagtctgttgacaccatgatccgtatggtgcgccgttattgggacgtccagggcaagcctgagaagaaaaccttgattggccgctggaatggttatcatggttccaccattggtggcgcatcgttgggcggtatgaaatacatgcacgaacagggcgatttgcctatcccaggtatggcccacattgagcagccttggtggtataagcacggtaaagatatgacccctgacgaatttggtgtggttgccgcacgctggttggaagaaaagatcttggagatcggtgccgataaggtcgcagcctttgttggcgagccaattcaaggtgcaggtggtgttatcgtcccacctgcaacctactggccagagatcgagcgtatttgccgtaagtatgacgttttgttggtcgccgatgaagtgatctgcggcttcggtcgcaccggtgagtggtttggtcaccaacactttggcttccagcctgacttgtttaccgccgcaaagggcttgtcttccggttacttgcctattggtgctgtttttgtgggtaaacgcgtcgcagaaggcttgattgccggtggcgactttaatcatggcttcacctactccggccatcctgtgtgcgcagcagtcgcacacgcaaatgtcgccgctttgcgcgacgaaggcattgtacagcgcgttaaagacgatattggtccatacatgcagaagcgctggcgtgaaaccttctctcgtttcgagcacgtcgatgatgtgcgtggcgttggtatggttcaagccttcactttggtcaaaaacaaggcaaagcgtgagttgtttcctgatttcggtgagatcggtaccttgtgccgtgacatctttttccgtaacaacttgattatgcgtgcatgtggtgatcacatcgtgtccgcaccacctttggttatgacccgtgccgaagtggacgagatgttggcagtggcagagcgctgtttggaagagtttgagcagaccttgaaagcccgtggtttggcctaa

SEQ ID NO: 12 - DNA, 1205 bp, Yarrowia lipolytica
  gene (1)..(1205): Ura3 gene
cgcccagagagccattgacgttctttctaatttggaccgatagccgtatagtccagtctatctataagttcaactaactcgtaactattaccataacatatacttcactgccccagataaggttccgataaaaagttctgcagactaaatttatttcagtctcctcttcaccaccaaaatgccctcctacgaagctcgagctaacgtccacaagtccgcctttgccgctcgagtgctcaagctcgtggcagccaagaaaaccaacctgtgtgcttctctggatgttaccaccaccaaggagctcattgagcttgccgataaggtcggaccttatgtgtgcatgatcaaaacccatatcgacatcattgacgacttcacctacgccggcactgtgctccccctcaaggaacttgctcttaagcacggtttcttcctgttcgaggacagaaagttcgcagatattggcaacactgtcaagcaccagtaccggtgtcaccgaatcgccgagtggtccgatatcaccaacgcccacggtgtacccggaaccggaatcattgctggcctgcgagctggtgccgaggaaactgtctctgaacagaagaaggaggacgtctctgactacgagaactcccagtacaaggagttcctagtcccctctcccaacgagaagctggccagaggtctgctcatgctggccgagctgtcttgcaagggctctctggccactggcgagtactccaagcagaccattgagcttgcccgatccgaccccgagtttgtggttggcttcattgcccagaaccgacctaagggcgactctgaggactggcttattctgacccccggggtgggtcttgacgacaagggagacgctctcggacagcagtaccgaactgttgaggatgtcatgtctaccggaacggatatcataattgtcggccgaggtctgtacggccagaaccgagatcctattgaggaggccaagcgataccagaaggctggctgggaggcttaccagaagattaactgttagaggttagactatggatatgtaatttaactgtgtatatagagagcgtgcaagtatggagcgcttgttcagcttgtatgatggtcagacgacctgtctgatcgagtatgtatgatactgcacaacctgtgtatccgcatgatctgtccaatggggcatgttgttgtgtttctcg

SEQ ID NO: 13 - DNA, 36 bp, Artificial Sequence
  BglII F primer for HisG1
aattgggcccagatctcagaccggttcagacaggat

SEQ ID NO: 14 - DNA, 36 bp, Artificial Sequence
  EcoRI R primer for HisG1
tctctgggcggaattcggaggtgcggatatgaggta

SEQ ID NO: 15 - DNA, 37 bp, Artificial Sequence
  NotI F primer for HisG1
tgtttctcggcggccgccagaccggttcagacaggat

SEQ ID NO: 16 - DNA, 36 bp, Artificial Sequence
  BamHI R primer for HisG1
tccaacgcgtggatccggaggtgcggatatgaggta

SEQ ID NO: 17 - DNA, 36 bp, Artificial Sequence
  BglII F primer for HisG2
aattgggcccagatctaacgctacctcgaccagaaa

SEQ ID NO: 18 - DNA, 36 bp, Artificial Sequence
  EcoRI R primer for HisG2
tctctgggcggaattctcttctcgatcggcagtacc

SEQ ID NO: 19 - DNA, 37 bp, Artificial Sequence
  NotI F primer for HisG2
tgtttctcggcggccgcaacgctacctcgaccagaaa

SEQ ID NO: 20 - DNA, 36 bp, Artificial Sequence
  BamHI R primer for HisG2
tccaacgcgtggatcctcttctcgatcggcagtacc

SEQ ID NO: 21 - DNA, 36 bp, Artificial Sequence
  BglII F primer for glt2
aattgggcccagatcttcagaacttgcgccgataaa

SEQ ID NO: 22 - DNA, 38 bp, Artificial Sequence
  EcoRI R primer for glt2
tctctgggcggaattcctttgccagctagaccatagag

SEQ ID NO: 23 - DNA, 37 bp, Artificial Sequence
  NotI F primer for glt2
tgtttctcggcggccgctcagaacttgcgccgataaa

SEQ ID NO: 24 - DNA, 38 bp, Artificial Sequence
  BamHI R primer for glt2
tccaacgcgtggatccctttgccagctagaccatagag

SEQ ID NO: 25 - DNA, 35 bp, Artificial Sequence
  BglII F primer for glt3
aattgggcccagatctattggcgggttcgttactt

SEQ ID NO: 26 - DNA, 38 bp, Artificial Sequence
  EcoRI R primer for glt3
tctctgggcggaattccctggaagaaggccgtattatc

SEQ ID NO: 27 - DNA, 36 bp, Artificial Sequence
  NotI F primer for glt3
tgtttctcggcggccgcattggcgggttcgttactt

SEQ ID NO: 28 - DNA, 38 bp, Artificial Sequence
  BamHI R primer for glt3
tccaacgcgtggatcccctggaagaaggccgtattatc

SEQ ID NO: 29 - DNA, 20 bp, Artificial Sequence
  F1 primer for ACO1
ttcctcaatggtggagaaga

SEQ ID NO: 30 - DNA, 46 bp, Artificial Sequence
  R1 primer for ACO1
tctttatcctgtctgaaccggtctggtaccatagtccttgccatgc

SEQ ID NO: 31 - DNA, 45 bp, Artificial Sequence
  F2 primer for ACO1
atcgctacctcatatccgcacctcccttctgtcccccgagtttct

SEQ ID NO: 32 - DNA, 19 bp, Artificial Sequence
  R2 primer for ACO1
aagaagggcttgagagtcg

SEQ ID NO: 33 - DNA, 17 bp, Artificial Sequence
  F1 primer for ACO2
cccaacaacactggcac

SEQ ID NO: 34 - DNA, 43 bp, Artificial Sequence
  R1 primer for ACO2
tctttatcctgtctgaaccggtctgctcctcatcgtagatggc

SEQ ID NO: 35 - DNA, 42 bp, Artificial Sequence
  F2 primer for ACO2
atcgctacctcatatccgcacctccgacaagacccgacaggc

SEQ ID NO: 36 - DNA, 18 bp, Artificial Sequence
  R2 primer for ACO2
agaccagagtcctcttcg

SEQ ID NO: 37 - DNA, 19 bp, Artificial Sequence
  F1 primer for ACO3
accttcacagagccaccca

SEQ ID NO: 38 - DNA, 36 bp, Artificial Sequence
  R1 primer for ACO3
atggctctctgggcggtgttgggggtgttgatgatg

SEQ ID NO: 39 - DNA, 36 bp, Artificial Sequence
  F2 primer for ACO3
ttgttgtgtttctcgcaaggttctcatcgaggcctg

SEQ ID NO: 40 - DNA, 22 bp, Artificial Sequence
  R2 primer for ACO3
aggaaaggtcgaagagtgctct

SEQ ID NO: 41 - DNA, 17 bp, Artificial Sequence
  F1 primer for ACO4
actgcgagagcgatctg

SEQ ID NO: 42 - DNA, 45 bp, Artificial Sequence
  R1 primer for ACO4
tctttatcctgtctgaaccggtctgttcatgagcatgtagtttcg

SEQ ID NO: 43 - DNA, 44 bp, Artificial Sequence
  F2 primer for ACO4
atcgctacctcatatccgcacctccgaggacgacaaagccggag

SEQ ID NO: 44 - DNA, 19 bp, Artificial Sequence
  R2 primer for ACO4
agagcagagtcctcctcaa

SEQ ID NO: 45 - DNA, 22 bp, Artificial Sequence
  F1 primer for ACO5
aacttcctcacaggcagcgagc

SEQ ID NO: 46 - DNA, 39 bp, Artificial Sequence
  R1 primer for ACO5
atggctctctgggcggagtagagagtgggagttgaggtc

SEQ ID NO: 47 - DNA, 34 bp, Artificial Sequence
  F2 primer for ACO5
ttgttgtgtttctcgccccgtcaaggacgctgag

SEQ ID NO: 48 - DNA, 22 bp, Artificial Sequence
  R2 primer for ACO5
acagtaaggtggggcttgactc

SEQ ID NO: 49 - DNA, 21 bp, Artificial Sequence
  F1 primer for ACO6
agtccctcaacacgtttaccg

SEQ ID NO: 50 - DNA, 46 bp, Artificial Sequence
  R1 primer for ACO6
tctttatcctgtctgaaccggtctgccatttagtggcagcaacgtt

SEQ ID NO: 51 - DNA, 45 bp, Artificial Sequence
  F2 primer for ACO6
atcgctacctcatatccgcacctccgagctctgatcaaccgaacc

SEQ ID NO: 52 - DNA, 20 bp, Artificial Sequence
  R2 primer for ACO6
aggaagggtctaatgacaga

SEQ ID NO: 53 - DNA, 17 bp, Artificial Sequence
  F1 primer for FALDH1
aatcactcctcctacgc

SEQ ID NO: 54 - DNA, 43 bp, Artificial Sequence
  R1 primer for FALDH1
tctttatcctgtctgaaccggtctgtggtctcggggacacctc

SEQ ID NO: 55 - DNA, 43 bp, Artificial Sequence
  F2 primer for FALDH1
atcgctacctcatatccgcacctccccatcatcaagccccgaa

SEQ ID NO: 56 - DNA, 20 bp, Artificial Sequence
  R2 primer for FALDH1
accgacataatctgagcaat

SEQ ID NO: 57 - DNA, 19 bp, Artificial Sequence
  F1 primer for FALDH2
accactaggtgagatcgag

SEQ ID NO: 58 - DNA, 45 bp, Artificial Sequence
  R1 primer for FALDH2
tctttatcctgtctgaaccggtctgctccgacactaccggaacgc

SEQ ID NO: 59 - DNA, 43 bp, Artificial Sequence
  F2 primer for FALDH2
atcgctacctcatatccgcacctcccttgctcccacagttgtt

SEQ ID NO: 60 - DNA, 19 bp, Artificial Sequence
  R2 primer for FALDH2
gatcacccagaaccatagc

SEQ ID NO: 61 - DNA, 19 bp, Artificial Sequence
  F1 primer for FALDH3
gtgacccccaccacgtcac

SEQ ID NO: 62 - DNA, 46 bp, Artificial Sequence
  R1 primer for FALDH3
tctttatcctgtctgaaccggtctgttctgacattttcagcgccac

SEQ ID NO: 63 - DNA, 45 bp, Artificial Sequence
  F2 primer for FALDH3
atcgctacctcatatccgcacctccccattacgagcgtttgacgg

SEQ ID NO: 64 - DNA, 17 bp, Artificial Sequence
  R2 primer for FALDH3
cagggctggggaccacc

SEQ ID NO: 65 - DNA, 19 bp, Artificial Sequence
  F1 primer for FALDH4
taccgactggaccagattc

SEQ ID NO: 66 - DNA, 46 bp, Artificial Sequence
  R1 primer for FALDH4
tctttatcctgtctgaaccggtctgcggcagtggcaatgatcttac

SEQ ID NO: 67 - DNA, 46 bp, Artificial Sequence
  F2 primer for FALDH4
atcgctacctcatatccgcacctccgactcgattcatcgctcctac

SEQ ID NO: 68 - DNA, 21 bp, Artificial Sequence
  R2 primer for FALDH4
caaatctttcggaagattcgg

SEQ ID NO: 69 - DNA, 20 bp, Artificial Sequence
  F primer for HISG1
cagaccggttcagacaggat

SEQ ID NO: 70 - DNA, 20 bp, Artificial Sequence
  R primer for HISG1
ggaggtgcggatatgaggta

SEQ ID NO: 71 - DNA, 20 bp, Artificial Sequence
  F primer for HISG2
aacgctacctcgaccagaaa

SEQ ID NO: 72 - DNA, 20 bp, Artificial Sequence
  R primer for HISG2
tcttctcgatcggcagtacc

SEQ ID NO: 73 - DNA, 20 bp, Artificial Sequence
  F primer for glt2
tcagaacttgcgccgataaa

SEQ ID NO: 74 - DNA, 22 bp, Artificial Sequence
  R primer for glt2
ctttgccagctagaccatagag

SEQ ID NO: 75 - DNA, 19 bp, Artificial Sequence
  F primer for glt3
attggcgggttcgttactt

SEQ ID NO: 76 - DNA, 22 bp, Artificial Sequence
  R primer for glt3
cctggaagaaggccgtattatc

SEQ ID NO: 77 - DNA, 23 bp, Artificial Sequence
  Ulura3 cs 2B primer for Bipartite
atgccctcctacgaagctcgagc

SEQ ID NO: 78 - DNA, 20 bp, Artificial Sequence
  Ylura3F primer for Bipartite
ctcccaacgagaagctggcc

SEQ ID NO: 79 - DNA, 42 bp, Artificial Sequence
  EXP1-F primer for transaminase vector
ccaagcttggtaccgagctcagagtttggcgcccgttttttc

SEQ ID NO: 80 - DNA, 42 bp, Artificial Sequence
  EXP1-R primer for transaminase vector
cgttgtttttgcatatgtgctgtagatatgtcttgtgtgtaa

SEQ ID NO: 81 - DNA, 42 bp, Artificial Sequence
  TEF-F primer for transaminase vector
ccaagcttggtaccgagctcaaactttggcaaagaggctgca

SEQ ID NO: 82 - DNA, 42 bp, Artificial Sequence
  TEF-R primer for transaminase vector
cgttgtttttgcatatgtttgaatgattcttatactcagaag

SEQ ID NO: 83 - DNA, 44 bp, Artificial Sequence
  ALK1-F primer for transaminase vector
ccaagcttggtaccgagctcagatctgtgcgcctctacagaccc

SEQ ID NO: 84 - DNA, 41 bp, Artificial Sequence
  ALK1-R primer for transaminase vector
cgttgtttttgcatatgagtgcaggagtattctggggagga

SEQ ID NO: 85 - DNA, 28 bp, Artificial Sequence
  XPR2t-F2 primer for transaminase vector
gtcgacgcaattaacagatagtttgccg

SEQ ID NO: 86 - DNA, 32 bp, Artificial Sequence
  XPR2t-R3 primer for transaminase vector
ctcgagggatcccggaaaacaaaacacgacag

SEQ ID NO: 87 - DNA, 28 bp, Artificial Sequence
  TA-F primer for transaminase vector
catatgcaaaaacaacgtactacctccc

SEQ ID NO: 88 - DNA, 28 bp, Artificial Sequence
  TA-R primer for transaminase vector
gtcgacttaggccaaaccacgggctttc

SEQ ID NO: 89 - DNA, 35 bp, Artificial Sequence
  ATATG2-ER-F primer for transaminase vector
actcctgcactcatatgtccaacgccctcaacctg

SEQ ID NO: 90 - DNA, 35 bp, Artificial Sequence
  XTATG2-ER-F primer for transaminase vector
ccaatccaacacatatgtccaacgccctcaacctg

SEQ ID NO: 91 - DNA, 82 bp, Artificial Sequence
  ER-R-1 primer for transaminase vector
cgttgtttttgcatagaaccgccaccgccgctaccgccaccgcccgaaccgccaccgccgaatcgtgaaatatccttgggct

SEQ ID NO: 92 - DNA, 85 bp, Artificial Sequence
  ER-R-2 primer for transaminase vector
cgttgtttttgcatatgagaaccgccaccgccgctaccgccaccgcccgaaccgccaccgccgaatcgtgaaatatccttgggct

SEQ ID NO: 93 - DNA, 37 bp, Artificial Sequence
  ETATG2-ER-1 primer for transaminase vector
tgattacgccaagcttgagtttggcgcccgttttttc

SEQ ID NO: 94 - DNA, 50 bp, Artificial Sequence
  ETATG2-ER-2 primer for transaminase vector
acaggttgagggcgttggacatatgtgctgtagatatgtcttgtgtgtaa

SEQ ID NO: 95 - DNA, 36 bp, Artificial Sequence
  TTATG2-ER-1 primer for transaminase vector
tgattacgccaagcttaaactttggcaaagaggctg

SEQ ID NO: 96 - DNA, 50 bp, Artificial Sequence
  TTATG2-ER-2 primer for transaminase vector
acaggttgagggcgttggacatatgtttgaatgattcttatactcagaag

SEQ ID NO: 97 - DNA, 21 bp, Artificial Sequence
  ER-F primer for transaminase vector
atgtccaacgccctcaacctg

SEQ ID NO: 98 - DNA, 34 bp, Artificial Sequence
  ER-R-3 primer for transaminase vector
cgttgtttttgcatagaaccgccaccgccgctac

SEQ ID NO: 99 - DNA, 19 bp, Artificial Sequence
  TA-FALDH4-F1 primer for transaminase vector
taccgactggaccagattc

SEQ ID NO: 100 - DNA, 21 bp, Artificial Sequence
  TA-FALDH4-R1 primer for transaminase vector
cggcagtggcaatgatcttac

SEQ ID NO: 101 - DNA, 45 bp, Artificial Sequence
  TA-FALDH4-F2 primer for transaminase vector
ctcctctatggtctagctggcaaagactcgattcatcgctcctac

SEQ ID NO: 102 - DNA, 21 bp, Artificial Sequence
  TA-FALDH4-R2 primer for transaminase vector
caaatctttcggaagattcgg

SEQ ID NO: 103 - DNA, 47 bp, Artificial Sequence
  ATATG2-F primer for transaminase vector
gtcggtaagatcattgccactgccgagatctgtgcgcctctacagac

SEQ ID NO: 104 - DNA, 46 bp, Artificial Sequence
  ETATG2-F primer for transaminase vector
gtcggtaagatcattgccactgccggagtttggcgcccgttttttc

SEQ ID NO: 105 - DNA, 46 bp, Artificial Sequence
  TTATG2-F primer for transaminase vector
gtcggtaagatcattgccactgccgaaactttggcaaagaggctgc

SEQ ID NO: 106 - DNA, 45 bp, Artificial Sequence
  XTATG2-F primer for transaminase vector
gtcggtaagatcattgccactgccgacgcgtggagagtttgggtt